(19) United States
(12) Patent Application Publication
(10) Pub. No.: US 2024/0279228 A1
(43) Pub. Date: Aug. 22, 2024

(54) NOVEL SALTS AND CRYSTALS

(71) Applicant: INTRA-CELLULAR THERAPIES, INC., New York, NY (US)

(72) Inventor: Peng LI, New Milford, NJ (US)

(21) Appl. No.: 18/567,743

(22) PCT Filed: Jun. 7, 2022
(86) PCT No.: PCT/US22/72802

§ 371 Date: Dec. 6, 2023

Related U.S. Application Data
(60) Provisional application No. 63/197,848, filed on Jun. 7, 2021.

(51) Int. Cl. C07D 471/16; A61K 31/4985

(52) U.S. Cl. CPC C07D 471/16; A61K 31/4985

(57) ABSTRACT

The disclosure provides new salts, crystals and co-crystal forms of particular substituted heterocycle fused gamma-carbolines, as described herein, together with methods of making and using them, and pharmaceutical compositions comprising them.

### CROSS-REFERENCE TO RELATED APPLICATIONS

This application is an international application which claims priority to and the benefit of U.S. Provisional Application No. 63/197,848, filed on Jun. 7, 2021, the contents of which are hereby incorporated by reference in its entirety.

### FIELD

This disclosure relates to certain salts, crystals and co-crystal forms of particular substituted heterocycle fused gamma-carbolines, as described herein, and the manufacture thereof, which are useful in the treatment of diseases involving the 5-HT_{2A} receptor, the serotonin transporter (SERT), pathways involving dopamine D₁ and/or D₂ receptor signaling systems, and/or the μ-opioid receptor.

### BACKGROUND

Substituted heterocycle fused gamma-carbolines are known to be agonists or antagonists of 5-HT₂ receptors, particularly 5-HT_{2A} receptors, in treating central nervous system disorders. These compounds have been disclosed in U.S. Pat. Nos. 6,548,493; 7,238,690; 6,552,017; 6,713,471; 7,183,282; U.S. RE39680, and U.S. RE39679, as novel compounds useful for the treatment of disorders associated with 5-HT_{2A} receptor modulation such as obesity, anxiety, depression, psychosis, schizophrenia, sleep disorders, sexual disorders migraine, conditions associated with cephalic pain, social phobias, gastrointestinal disorders such as dysfunction of the gastrointestinal tract motility, and obesity. U.S. Pat. Nos. 8,309,722, and 7,081,455, also disclose methods of making substituted heterocycle fused gamma-carbolines and uses of these gamma-carbolines as serotonin agonists and antagonists useful for the control and prevention of central nervous system disorders such as addictive behavior and sleep disorders. U.S. Pat. Nos. 8,648,077, 9,199,995, and 9,586,960, also disclose certain solid, crystalline salt forms of such compounds. Additional crystalline forms of such compounds are disclosed, for example, in US 2019/0112309, US 2019/0112310, US 2020/0247805, and US 2020/0157100.

More recently, newer substituted oxo-fused gamma carbolines have been disclosed which retain much of the unique pharmacologic activity of the previously disclosed compounds, including serotonin receptor inhibition, SERT inhibition, and dopamine receptor modulation, but with unexpectedly potent activity at mu-opioid receptors. Such compounds have been disclosed, for example, in U.S. Pat. Nos. 10,245,260, 10,799,500, US 2019/0330211, US 2019/0345160, US 2021/0145829, and US 2021/0163481, the contents of each of which are incorporated herein by reference in their entireties.

For example, the Compound A, shown below, is a potent serotonin 5-HT_{2A} receptor antagonist and mu-opioid receptor partial agonist or biased agonist. This compound also interacts with dopamine receptors, in particular the dopamine D1 receptors.

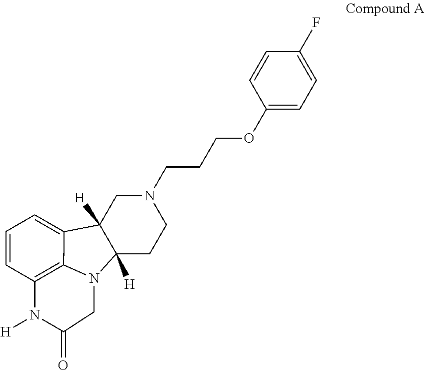 [SMILES: O=C1CN2c3c(cccc3[C@@H]3CN(CCCOc4ccc(F)cc4)CC[C@@H]32)N1]

It is also believed that the Compound A, via its D1 receptor activity, may also enhance NMDA and AMPA mediated signaling through the mTOR pathway. The Compound A is thus useful for the treatment or prophylaxis of central nervous system disorders, but there is a need in the art additional compounds having this unique biochemical and pharmacological profile, especially those which may have subtly altered pharmacologic or pharmacokinetic profiles compared to the Compound A.

The preparation of substituted heterocycle fused gamma-carbolines in free or pharmaceutically acceptable salt forms, intermediates used in their preparation, for example enantiomerically pure 2,3,4,4a,5,9b-hexahydro-1H-pyrido[4,3-b]indole type intermediates, and methods for producing said intermediates and said substituted heterocycle fused gamma-carbolines are disclosed in U.S. Pat. Nos. 7,183,282, 8,309,722, 8,779,139, 9,315,504, and 9,751,883, the entire contents of each of which are hereby incorporated by reference.

In addition, methods of preparing particular fused gamma-carbolines in high purity, yield and economic efficiency are disclosed in WO 2020/131895 and US 2022/0041600. In US 2017/0319580 and U.S. Pat. No. 10,799,500, a preparation of the compound in free base form is demonstrated. Further studies on this free base compound have shown that it can form crystals which may undesirably entrap organic solvent. See WO2020/131895. Only one prior salt of Compound A has been specifically demonstrated. A solid tosylate salt is also demonstrated in WO2020/131895, which discolors on standing but shows acceptable crystallinity.

There remains a need for pure, stable, solid, crystalline forms of such compounds in order to provide the most stable and reliable pharmaceutical formulations, with predictable and reproducible bioavailability, especially for oral formulations.

### BRIEF SUMMARY

In an effort to find new salts and polymorphs of the Compound A, an extensive salt screen was undertaken. The Compound A has very poor solubility in free base form. It was found to form a brown crystalline powder with a melting event at Tpeak=145° C. The free base compound exists as agglomerated blocks of less than 20 μm in size, and is slightly hygroscopic. The compound is freely soluble in DMSO, sparingly soluble in ethyl salicylate and anisole, and less than sparingly soluble in most organic solvents and water.

Unexpectedly, it was found that the Compound A does not readily form salts reproducibly with common, pharmaceutically acceptable acids.

A major salt screen was carried out, wherein the free base compound was studied in different solvent systems and under different conditions, and then systematically screened using a selection of 58 acids under different conditions and with different solvents, co-solvents and anti-solvent systems, to identify new possible salt forms. In addition, a co-crystal screen using a variety of conditions using 18 amino acid co-formers was conducted. Following this extensive screening and experimentation, it was found that the most promising crystalline forms of Compound A are the toluenesulfonate salt, an alanine co-crystal and a phenylalanine co-crystal.

The present disclosure thus provides new forms of Compound A, which are especially advantageous for use in the preparation of galenic formulations, together with methods of making and using the same.

Further areas of applicability of the present invention will become apparent from the detailed description provided hereinafter. It should be understood that the detailed description and specific examples, while indicating preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings.

FIG. 1 depicts an X-ray powder diffraction (XRPD) pattern for the Compound A toluenesulfonic acid salt of Example 3.

FIG. 2 depicts TGA-DSC thermograms of the Compound A toluenesulfonic acid salt of Examples 2-B (top) and 2-A (bottom).

FIG. 3 depicts a TGA-DSC thermogram of the Compound A toluenesulfonic acid salt of Example 3.

FIG. 4 depicts an X-ray powder diffraction (XRPD) pattern for the Compound A toluenesulfonic acid salt of Example 5.

FIG. 5 depicts a DSC thermogram of the Compound A toluenesulfonic acid salt of Example 5.

FIG. 6 depicts a TG thermogram of the Compound A toluenesulfonic acid salt of Example 5.

FIG. 7 depicts an X-ray powder diffraction (XRPD) pattern for the Compound A oxalic acid salt.

FIG. 8 depict TGA-DSC thermograms of the Compound A oxalic acid salt of Examples 2-E (top) and 2-D (bottom).

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

In a first embodiment, the invention provides (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one (Compound A) in solid, crystalline salt form (Salt 1), wherein the salt form is selected from a hydrochloride, a p-toluenesulfonate, a tartrate, a malate, a fumarate, a glutamate, an oxalate, a besylate, and an ascorbate, optionally wherein the salt is chemically stable in air (e.g., does not undergo physical or chemical changes, such as appearance or color changes).

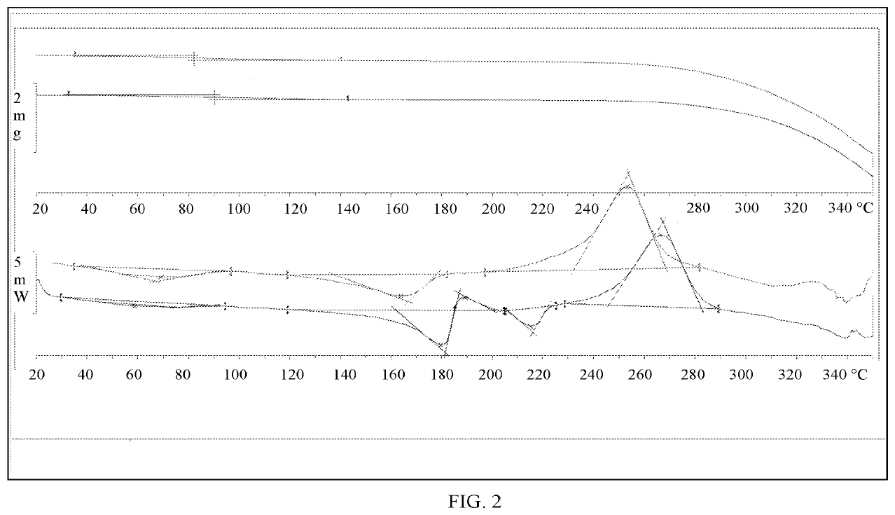

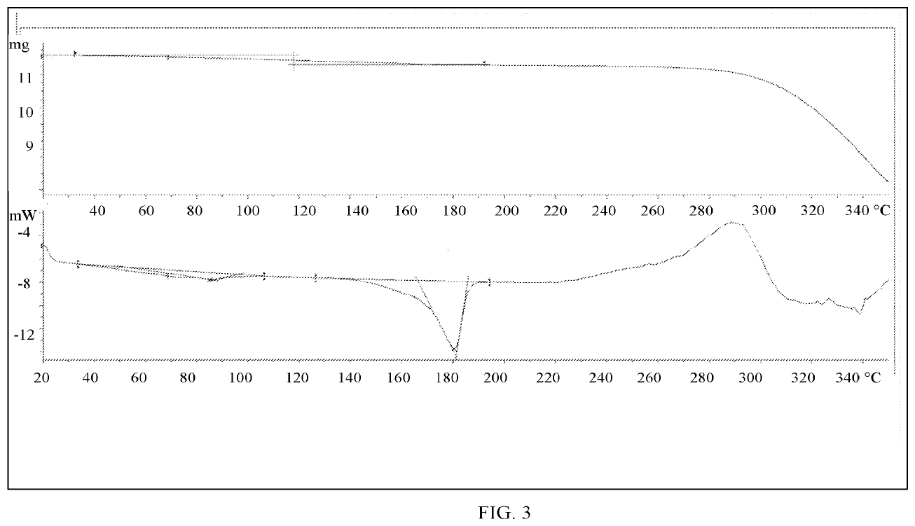

In additional embodiments, the present disclosure provides the following:
1.1. Salt 1, wherein the salt is at least 90% pure, e.g., at least 92%, at least 94%, at least 95%, at least 96%, at least 97%, at least 98% or at least 99% pure, for example, as shown by LCMS.
1.2. Salt 1 or 1.1, wherein the salt comprises less than 10 wt. %, e.g., less than 8 wt. %, or less than 6 wt. %, or less than 5 wt. %, or less than 4 wt. %, or less than 3 wt. %, or less than 2 wt. %, or less than 1 wt. %, or less than 0.5 wt. %, or less than 0.1 wt. %, of Compound A in free base form.
1.3. Salt 1 or any of 1.1-1.2, wherein the salt comprises less than 10 wt. %, e.g., less than 8 wt. %, or less than 6 wt. %, or less than 5 wt. %, or less than 4 wt. %, or less than 3 wt. %, or less than 2 wt. %, or less than 1 wt. %, or less than 0.5 wt. %, or less than 0.1 wt. %, of Compound A in any other salt form.
1.4. Salt 1 or any of 1.1-1.3, wherein the salt comprises less than 10 wt. %, e.g., less than 8 wt. %, or less than 6 wt. %, or less than 5 wt. %, or less than 4 wt. %, or less than 3 wt. %, or less than 2 wt. %, or less than 1 wt. %, or less than 0.5 wt. %, or less than 0.1 wt. %, of any p-toluenesulfonic acid, tartaric acid, malic acid, fumaric acid, glutamic acid, oxalic acid, benzenesulfonic acid, or ascorbic acid.
1.5. Salt 1 or any of 1.1-1.4, wherein the salt comprises less than 10 wt. %, e.g., less than 8 wt. %, or less than 6 wt. %, or less than 5 wt. %, or less than 4 wt. %, or less than 3 wt. %, or less than 2 wt. %, or less than 1 wt. %, or less than 0.5 wt. %, or less than 0.1 wt. %, of any free solvent (e.g., other than solvent comprised in solvated crystal forms).
1.6. Salt 1 or any of 1.1-1.5, wherein the salt comprises less than 10 wt. %, e.g., less than 8 wt. %, or less than 6 wt. %, or less than 5 wt. %, or less than 4 wt. %, or less than 3 wt. %, or less than 2 wt. %, or less than 1 wt. %, or less than 0.5 wt. %, or less than 0.1 wt. %, of any synthetic precursor of, or side-product from, the synthetic method used to prepare the Compound A in free base form.
1.7. Salt 1 or any of 1.1-1.6, wherein the salt is in a homogeneous crystal form, e.g., free or substantially free of other crystalline forms or polymorphs or amorphous forms of Compound A, e.g., free or substantially free, e.g., less than 10 wt. %, preferably less than about 5 wt. %, more preferably less than about 2 wt. %, still more preferably less than about 1 wt. %, still more preferably less than about 0.1%, and most preferably less than about 0.01 wt. %, of any other crystalline forms or polymorph forms or amorphous forms.
1.8. Salt 1, or any of 1.1-1.7, wherein the salt is a p-toluenesulfonic acid salt.
1.9. Salt 1, or any of 1.1-1.7, wherein the salt is a hydrochloric acid salt.
1.10. Salt 1, or any of 1.1-1.7, wherein the salt is a tartaric acid salt.
1.11. Salt 1, or any of 1.1-1.7, wherein the salt is a malic acid salt.
1.12. Salt 1, or any of 1.1-1.7, wherein the salt is a fumaric acid salt.
1.13. Salt 1, or any of 1.1-1.7, wherein the salt is a glutamic acid salt.
1.14. Salt 1, or any of 1.1-1.7, wherein the salt is an oxalic acid salt.
1.15. Salt 1, or any of 1.1-1.7, wherein the salt is an ascorbic acid salt.
1.16. Salt 1, or any of 1.1-1.7, wherein the salt is a benzenesulfonic acid salt.
1.17. Salt 1, or any of 1.1-1.16, wherein the salt is a salt comprising the Compound A free base and the acid counterion (e.g., p-toluenesulfonate anion) in a 1:1 molar ratio (i.e., a monotosylate salt).
1.18. Salt 1, or any of 1.1-1.16, wherein the salt is a salt comprising the Compound A free base and the acid counterion (e.g., p-toluenesulfonate anion) in a 1:2 or 1:3 molar ratio.
1.19. Salt 1, or any of 1.1-1.18, wherein the salt is formed by reacting Compound A in free base form with the counter-acid (e.g., p-toluenesulfonic acid) in a 1:0.9 to 1:5 molar ratio, e.g., 1:0.9 to 1:3, or 1:0.9 to 1:1.5, or 1:0.9 to 1:1.2, or 1:0.9 to 1:1.1, or about 1:1.
1.20. Salt 1, or any of 1.1-1.19, wherein the salt is formed by reacting the Compound A in free base form with the counter-acid (e.g., p-toluenesulfonic acid) in a solvent selected from methanol, dimethyl sulfoxide, acetone, acetonitrile, ethyl acetate, toluene, 2-butanone, benzonitrile, cyclohexane, tetrahydrofuran, and diisopropyl ether, or a mixture thereof, optionally further comprising water or hexane.
1.21. Any foregoing form of Salt 1, wherein the salt is a solvate, e.g., a solvate selected from a methanol, dimethyl sulfoxide, acetone, acetonitrile, ethyl acetate, toluene, or 2-butanone solvate.
1.22. Any foregoing form of Salt 1, wherein the salt is not a solvate.
1.23. Any foregoing form of Salt 1, wherein the salt is a hydrate.
1.24. Any foregoing form of Salt 1, wherein the salt is not a hydrate.
1.25. Any foregoing form of Salt 1, wherein the salt is a toluenesulfonic acid addition salt prepared from (e.g., crystallized from) acetone, ethyl acetate, toluene or 2-butanone solvent.
1.26. Any foregoing form of Salt 1, wherein the salt is a toluenesulfonic acid salt prepared from (e.g., crystallized from) acetone or 2-butanone solvent.
1.27. Any foregoing form of Salt 1, wherein the salt is a toluenesulfonic acid salt, optionally wherein the salt was prepared in (e.g., crystallized from) 2-butanone solvent, the salt having a DSC/TGA thermogram showing one endothermic event at about 40-100° C. (e.g., about 67° C.), a second endothermic event at about 120-180° C. (e.g., about 164° C.), and one exothermic event at about 200-280° C. (e.g., about 256° C.), e.g., wherein the first endothermic event is a desolvation event and/or the second endothermic event is a melt of the salt, and/or wherein the exothermic event is a decomposition.
1.28. Any foregoing form of Salt 1, wherein the salt is a toluenesulfonic acid salt, optionally wherein the salt was prepared in (e.g., crystallized from) acetone solvent, the salt having a DSC/TGA thermogram showing one endothermic event at about 40-100° C. (e.g., about 56° C.), a second endothermic event at about 120-190° C. (e.g., about 180° C.), a third endothermic event at about 200-220° C. (e.g., about 216° C.), and one exothermic event at about 230-290° C. (e.g., about 269° C.), e.g., wherein the first endothermic event is a desolvation event and/or the second endothermic event is a melt of the salt, and/or wherein the exothermic event is a decomposition.
1.29. Any foregoing form of Salt 1, wherein the salt is a toluenesulfonic acid salt, optionally wherein the salt was prepared in (e.g., crystallized from) 2-butanone solvent, the salt having a DSC/TGA thermogram showing one endothermic event at about 84° C., and a second exothermic event at about 180° C., e.g., wherein the DSC/TGA thermogram shows the first endothermic event at about Tₒₙₛₑₜ=49° C., Tₚₑₐₖ=84° C. and ΔE=−13 J/g, and the second endothermic event at about Tₒₙₛₑₜ=166° C., Tₚₑₐₖ=180° C. and ΔE=−47 J/g, for example, wherein the first endothermic event is a desolvation and the second endothermic event is a melt.
1.30. Any foregoing form of Salt 1, wherein the salt is a toluenesulfonic acid salt, optionally wherein the salt was prepared in (e.g., crystallized from) 2-butanone solvent or acetone solvent, the salt having a DSC/TGA thermogram as shown in FIG. 2 or FIG. 3.
1.31. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern corresponding to the d-spacing and/or angle (2-theta) values of the following table, for example, wherein the pattern comprises at least five, or at least six, or at least seven, or at least eight of said values, e.g., taking into account potential variations due to sample purity and instrument variation, for example 2θ shifts due to variation in X-ray wavelength, e.g., wherein the X-ray powder diffraction pattern is generated using an X-ray diffractometer with a copper anode and a nickel filter, e.g., comprising at least those peaks having a relative intensity of at least 0.4, at least 0.5, or at least 0.6, or comprising peaks 5, 6, 7, 9, 10, and 12:

XRPD (Cu anode, Ni filter) for p-Tosylate Salt Crystal Polymorph 1

| # | Angle | d Value | Rel. Intensity |
|---|---|---|---|
| 1 | 5.877 | 15.02674 | 7.20 |
| 2 | 8.627 | 10.24128 | 1.50 |
| 3 | 11.437 | 7.73074 | 4.20 |
| 4 | 13.545 | 6.53209 | 10.80 |
| 5 | 14.933 | 5.92793 | 89.70 |
| 6 | 16.975 | 5.21919 | 63.00 |
| 7 | 19.371 | 4.5787 | 100.00 |
| 8 | 19.957 | 4.44545 | 30.60 |
| 9 | 20.94 | 4.23896 | 24.20 |
| 10 | 22.592 | 3.93251 | 18.80 |
| 11 | 23.807 | 3.7346 | 9.90 |
| 12 | 24.567 | 3.62067 | 44.00 |
| 13 | 25.342 | 3.51164 | 15.90 |
| 14 | 25.606 | 3.47614 | 16.90 |
| 15 | 25.944 | 3.4316 | 11.70 |
| 16 | 27.532 | 3.23719 | 11.70 |
| 17 | 28.041 | 3.17951 | 1.80 |
| 18 | 28.968 | 3.07987 | 3.60 |
| 19 | 29.943 | 2.98173 | 6.00 |
| 20 | 32.527 | 2.75052 | 4.60 |
| 21 | 33.612 | 2.66415 | 2.80 |
| 22 | 36.548 | 2.45664 | 3.60 |
| 23 | 36.588 | 2.45405 | 4.30 |
| 24 | 36.615 | 2.45229 | 3.80 |

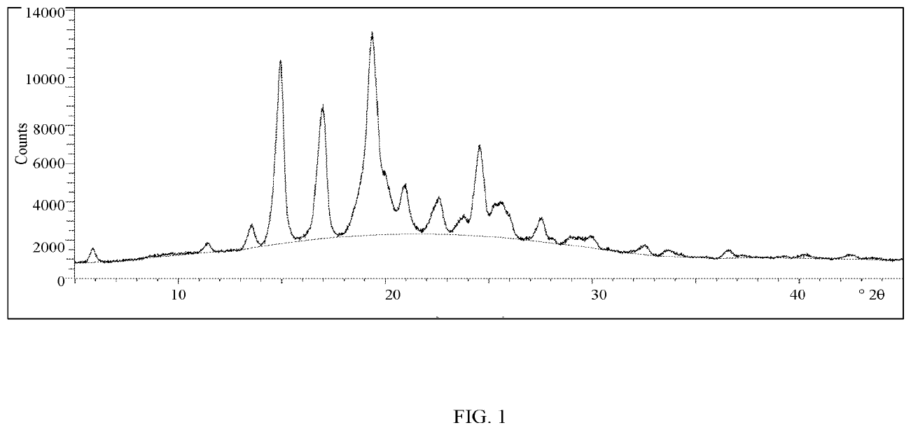

1.32. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern corresponding to FIG. 1, e.g., taking into account potential variations due to sample purity and instrument variation, for example 2θ shifts due to variation in X-ray wavelength, e.g., an X-ray powder diffraction pattern corresponding to FIG. 1 generated using an X-ray diffractometer with a copper anode and a nickel filter.
1.33. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern having at least 5, or at least 6, or at least 7, or at least 8, peaks having angle (2-theta) values selected from the group consisting of about 5.9, 8.6, 11.4, 13.6, 14.9, 17.0, 19.4, 20.0, 20.9, 22.6, 23.8, 24.6, 25.3, 25.6, 25.9, 27.5, 28.0, 29.0, 29.9, 32.5, 33.6, 36.6, 36.6, and 36.6, taking into account potential variations due to sample purity and instrument variation, e.g., wherein the X-ray powder diffraction pattern is generated using an X-ray diffractometer with a copper anode and a nickel filter.
1.34. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern having at least 5, or at least 6, or at least 7, or at least 8, peaks having angle (2-theta) values selected from the group consisting of about 5.88, 8.63, 11.44, 13.55, 14.93, 16.98, 19.37, 19.96, 20.94, 22.59, 23.81, 24.57, 25.34, 25.61, 25.94, 27.53, 28.04, 28.97, 29.94, 32.53, 33.61, 36.55, 36.59, and 36.62, taking into account potential variations due to sample purity and instrument variation, e.g., wherein the X-ray powder diffraction pattern is generated using an X-ray diffractometer with a copper anode and a nickel filter.
1.35. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern at least 5, or at least 6, or at least 7, or at least 8, peaks having d-spacing values selected from the group consisting of about 15.03, 10.24, 7.73, 6.53, 5.93, 5.22, 4.58, 4.45, 4.24, 3.93, 3.74, 3.62, 3.51, 3.48, 3.43, 3.24, 3.18, 3.08, 2.98, 2.75, 2.66, 2.46, 2.454, and 2.452, taking into account potential variations due to sample purity and instrument variation, wherein the X-ray powder diffraction pattern is generated using an X-ray diffractometer with a copper anode and a nickel filter.
1.36. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern at least 5, or at least 6, or at least 7, or at least 8, peaks having d-spacing values selected from the group consisting of about 15.0, 10.2, 7.7, 6.5, 5.9, 5.2, 4.6, 4.5, 4.2, 3.9, 3.7, 3.6, 3.5, 3.5, 3.4, 3.2, 3.2, 3.1, 3.0, 2.8, 2.7, 2.5, 2.5, and 2.5, taking into account potential variations due to sample purity and instrument variation, wherein the X-ray powder diffraction pattern is generated using an X-ray diffractometer with a copper anode and a nickel filter.
1.37. Any foregoing form of Salt 1, wherein the salt is a toluenesulfonic acid salt, optionally wherein the salt was prepared in (e.g., crystallized from) 2-butanone solvent or acetone solvent, the salt having an X-ray powder diffraction pattern as shown, or substantially as shown, in FIG. 1.
1.38. Any foregoing form of Salt 1, wherein the salt is a toluenesulfonic acid salt, optionally wherein the salt was prepared in 2-butanone solvent or in acetone solvent, the salt having a crystal structure characterized by an X-ray powder diffraction pattern corresponding to the d-spacing and/or angle (2-theta) values of the following table, for example, wherein the pattern comprises at least five, or at least six, or at least seven, or at least eight of said values, e.g., taking into account potential variations due to sample purity and instrument variation, for example 20 shifts due to variation in X-ray wavelength, e.g., wherein the X-ray powder diffraction pattern is generated using an X-ray diffractometer with a copper anode and a nickel filter, e.g., comprising at least those peaks having a relative intensity of at least 0.4, at least 0.5, or at least 0.6, or comprising peaks 1, 2, 3, 4, 5, 6, 8, 10, 11, 12, 13, 21, 23, and 27:

XRPD (Cu anode, Ni filter) for p-Tosylate Salt Crystal Polymorph 2

| # | Angle | d Value | Rel. Intensity |
|---|---|---|---|
| 1 | 2.8879 | 30.59400 | 59.95 |
| 2 | 5.6562 | 15.62516 | 100.00 |
| 3 | 8.4644 | 10.44645 | 8.16 |
| 4 | 11.2928 | 7.83563 | 26.92 |
| 5 | 13.4082 | 6.60377 | 9.39 |
| 6 | 14.8248 | 5.97578 | 78.72 |
| 7 | 16.6241 | 5.33282 | 16.49 |
| 8 | 16.9582 | 5.22852 | 90.26 |
| 9 | 18.4273 | 4.81487 | 5.19 |
| 10 | 18.6759 | 4.75131 | 19.75 |
| 11 | 19.1869 | 4.62591 | 32.80 |
| 12 | 19.4590 | 4.56184 | 18.15 |
| 13 | 19.8260 | 4.47822 | 48.35 |
| 14 | 20.2312 | 4.38943 | 6.11 |
| 15 | 20.8301 | 4.26457 | 16.06 |
| 16 | 22.1645 | 4.01075 | 3.91 |
| 17 | 22.5080 | 3.95030 | 10.90 |
| 18 | 22.6768 | 3.92128 | 9.65 |
| 19 | 23.3069 | 3.81667 | 6.41 |
| 20 | 23.6713 | 3.75874 | 9.16 |
| 21 | 24.5094 | 3.63207 | 25.32 |
| 22 | 25.0812 | 3.55056 | 6.40 |
| 23 | 25.5465 | 3.48693 | 24.59 |
| 24 | 25.8673 | 3.44441 | 6.35 |
| 25 | 26.8693 | 3.31820 | 4.06 |
| 26 | 27.4048 | 3.25187 | 11.53 |
| 27 | 27.4701 | 3.24697 | 14.54 |
| 28 | 27.9988 | 3.18686 | 3.56 |
| 29 | 28.4245 | 3.14009 | 2.31 |
| 30 | 28.8342 | 3.09640 | 2.51 |
| 31 | 29.2912 | 3.04911 | 2.79 |
| 32 | 29.9137 | 2.98706 | 2.90 |
| 33 | 31.3520 | 2.85324 | 5.46 |
| 34 | 32.4971 | 2.75526 | 3.68 |
| 35 | 33.7432 | 2.65632 | 3.64 |
| 36 | 34.2165 | 2.62065 | 2.52 |
| 37 | 36.5083 | 2.46123 | 6.15 |
| 38 | 37.3422 | 2.40816 | 1.74 |
| 39 | 37.9031 | 2.37381 | 2.00 |
| 40 | 39.2907 | 2.29311 | 1.56 |

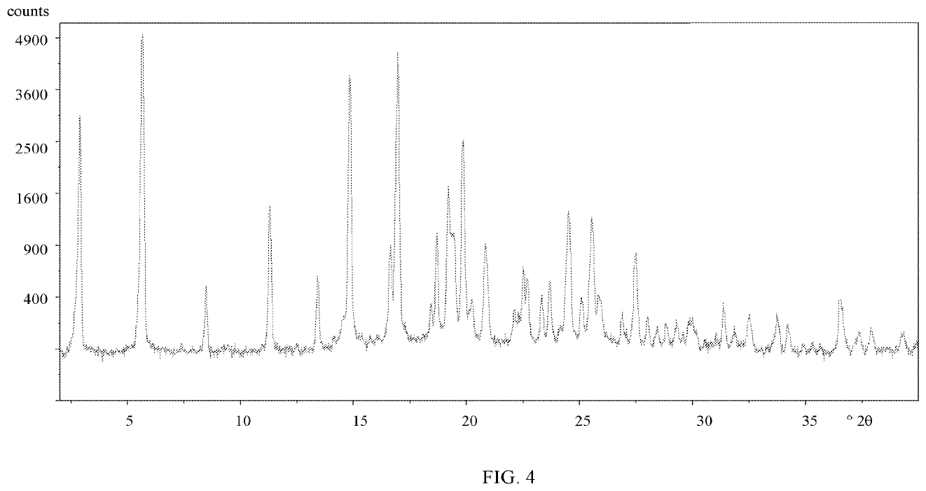

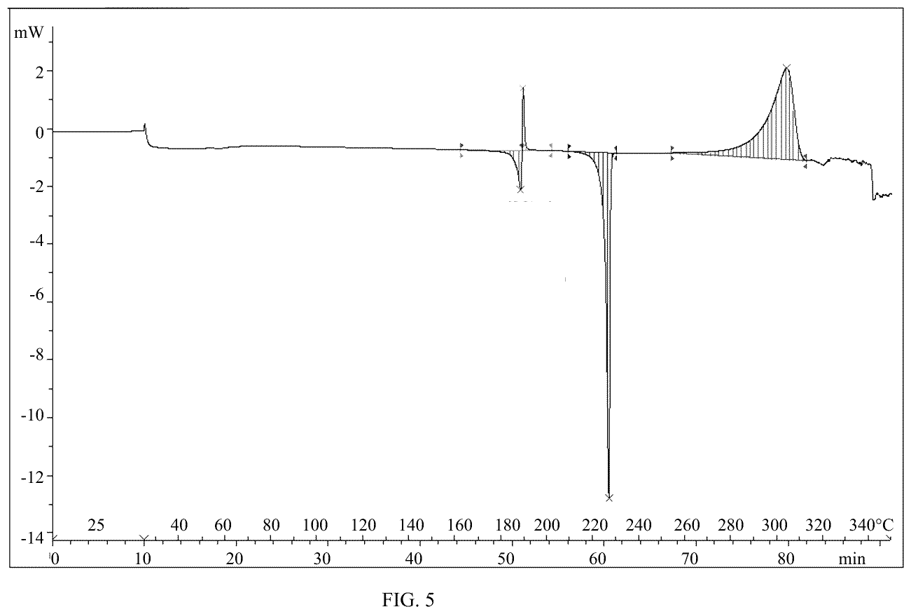

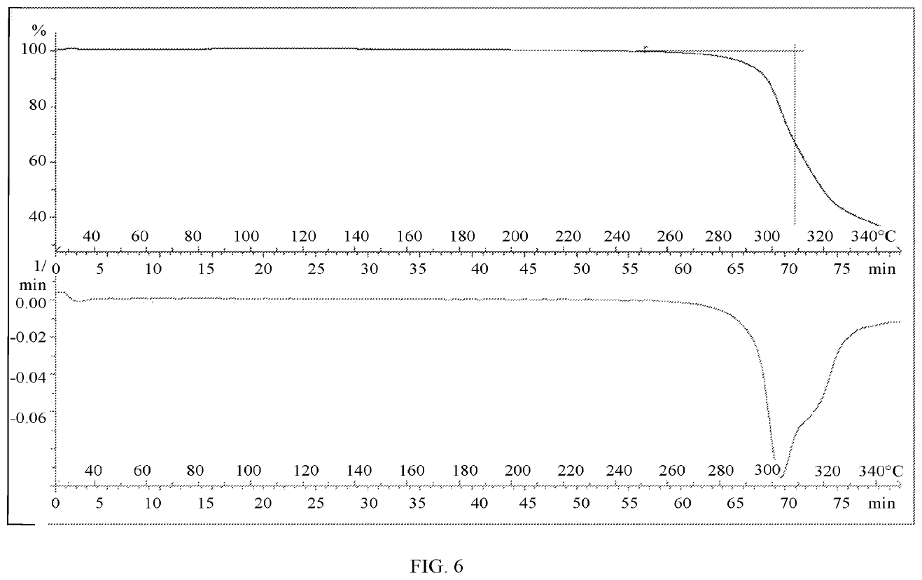

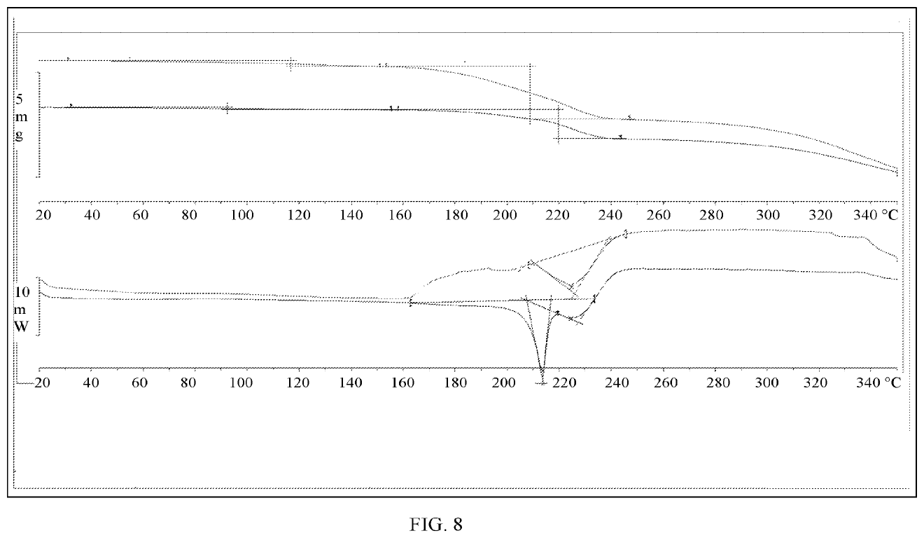

1.39. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern corresponding to FIG. 4, e.g., taking into account potential variations due to sample purity and instrument variation, for example 2θ shifts due to variation in X-ray wavelength, e.g., an X-ray powder diffraction pattern corresponding to FIG. 1 generated using an X-ray diffractometer with a copper anode and a nickel filter.
1.40. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern having at least 5, or at least 6, or at least 7, or at least 8, peaks having angle (2-theta) values selected from the group consisting of about 2.9, 5.7, 8.5, 11.3, 13.4, 14.8, 16.6, 17.0, 18.4, 18.7, 19.2, 19.5, 19.8, 20.2, 20.8, 22.2, 22.5, 22.7, 23.3, 23.7, 24.5, 25.1, 25.6, 25.9, 26.9, 27.4, 27.5, 28.0, 28.4, 28.8, 29.9, 31.4, 32.5, 33.7, 34.2, 36.5, 37.3, 37.9, and 39.3, taking into account potential variations due to sample purity and instrument variation, e.g., wherein the X-ray powder diffraction pattern is generated using an X-ray diffractometer with a copper anode and a nickel filter.
1.41. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern having at least 5, or at least 6, or at least 7, or at least 8, peaks having angle (2-theta) values selected from the group consisting of about 2.89, 5.66, 8.46, 11.29, 13.41, 14.82, 16.62, 16.96, 18.43, 18.68, 19.19, 19.46, 19.83, 20.23, 20.83, 22.16, 22.51, 22.68, 23.31, 23.67, 24.51, 25.08, 25.55, 25.87, 26.87, 27.41, 27.47, 28.00, 28.43, 28.83, 29.92, 31.35, 32.50, 33.74, 34.22, 36.51, 37.34, 37.90, and 39.29, taking into account potential variations due to sample purity and instrument variation, e.g., wherein the X-ray powder diffraction pattern is generated using an X-ray diffractometer with a copper anode and a nickel filter.
1.42. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern at least 5, or at least 6, or at least 7, or at least 8, peaks having d-spacing values selected from the group consisting of about 30.59, 15.63, 10.45, 7.84, 6.60, 5.98, 5.33, 5.23, 4.81, 4.75, 4.63, 4.56, 4.48, 4.39, 4.26, 4.01, 3.95, 3.92, 3.82, 3.76, 3.63, 3.55, 3.49, 3.44, 3.32, 3.252, 3.247, 3.19, 3.14, 3.10, 3.05, 2.99, 2.85, 2.76, 2.66, 2.62, 2.46, 2.41, 2.37, and 2.29, taking into account potential variations due to sample purity and instrument variation, wherein the X-ray powder diffraction pattern is generated using an X-ray diffractometer with a copper anode and a nickel filter.
1.43. Any foregoing form of Salt 1, wherein the salt is a toluenesulfonic acid salt, optionally wherein the salt was prepared in (e.g., crystallized from) 2-butanone solvent or acetone solvent, the salt having a crystal structure characterized by an X-ray powder diffraction pattern at least 5, or at least 6, or at least 7, or at least 8, peaks having d-spacing values selected from the group consisting of about 30.6, 15.6, 10.5, 7.8, 6.6, 6.0, 5.3, 5.2, 4.8, 4.8, 4.6, 4.6, 4.5, 4.4, 4.3, 4.0, 4.0, 3.9, 3.8, 3.8, 3.6, 3.6, 3.5, 3.4, 3.3, 3.3, 3.3, 3.2, 3.1, 3.1, 3.1, 3.0, 2.9, 2.8, 2.7, 2.6, 2.5, 2.4, 2.4, and 2.3, taking into account potential variations due to sample purity and instrument variation, wherein the X-ray powder diffraction pattern is generated using an X-ray diffractometer with a copper anode and a nickel filter.
1.44. Any foregoing form of Salt 1, wherein the salt is a toluenesulfonic acid salt, optionally wherein the salt was prepared in (e.g., crystallized from) 2-butanone solvent or acetone solvent, the salt having an X-ray powder diffraction pattern as shown, or substantially as shown, in FIG. 4.
1.45. Any foregoing form of Salt 1 (e.g., any of Salts 1.38-1.44), wherein the salt is a toluenesulfonic acid salt, optionally wherein the salt was prepared in (e.g., crystallized from) 2-butanone solvent, the salt having a DSC thermogram showing one endothermic event at about 186-190° C. (e.g., peak 188° C.), a sharp exothermic event at about 189-202° C. (e.g., peak 189° C.), a second endothermic event at about 225-230° C. (e.g., peak 226° C.), and a second exothermic event at about 260-310° C. (e.g., peak 304° C.), e.g., wherein the first endothermic event is a melting event, the first exothermic event is a recrystallization, the second endothermic event is a melting event, and the second exothermic event is a decomposition, and optionally, wherein the salt has a TG thermogram showing a single mass loss beginning at about 250° C.
1.46. Any foregoing form of Salt 1, wherein the salt is a toluenesulfonic acid salt, optionally wherein the salt was prepared in (e.g., crystallized from) 2-butanone solvent or acetone solvent, the salt having a DSC thermogram as shown in FIG. 5 and/or a TGA thermogram as shown in FIG. 6.
1.47. Any foregoing form of Salt 1, wherein the salt is a toluenesulfonic acid salt, optionally wherein the salt was prepared in (e.g., crystallized from) 2-butanone solvent or acetone solvent, and wherein the salt has a melting point of about 163° C.
1.48. Any foregoing form of Salt 1, wherein the salt is a toluenesulfonic acid salt, optionally wherein the salt was prepared in (e.g., crystallized from) 2-butanone solvent or acetone solvent, and wherein the salt crystals have a monoclinic form.
1.49. Any foregoing form of Salt 1, wherein the salt is a toluenesulfonic acid salt, optionally wherein the salt was prepared in (e.g., crystallized from) 2-butanone solvent or acetone solvent, the salt having an X-ray powder diffraction pattern relative angle (2-theta) values as provided in any one of embodiments 1.31 to 1.44 above, wherein the values are shifted by up to +/−0.2 degrees, e.g., wherein the values are substantially uniformly shifted by up to +/−0.2 degrees.
1.50. Any one of Salts 1.26-1.49, wherein the salt is prepared in acetone solvent.
1.51. Salt 1.50, wherein the salt is prepared by combining one equivalent of Compound A free base with 0.95 to 1.25 equivalents of p-toluenesulfonic acid (e.g., p-toluenesulfonic acid monohydrate) in acetone solvent, optionally wherein the p-toluenesulfonic acid is added to the reaction as a solution in acetone solvent.
1.52. Salt 1.51, wherein the reaction comprises 0.95 to 1.15 equivalents of p-toluenesulfonic acid (e.g., p-toluenesulfonic acid monohydrate), or 0.95 to 1.10 equivalents, or 0.95 to 1.05 equivalents, or 0.95 to 1.00 equivalents, or 0.95 to 0.99 equivalents.
1.53. Any of salts 1.50-1.52, wherein the salt is prepared at a temperature of 0° C. to 40° C., e.g., 0° C. to 30° C., or 0° C. to 25° C., or 0° C. to 15° C., or 5° C. to 20° C., or 5° C. to 15° C., or 10° C. to 20° C., or 10° C. to 15° C., or 5° C. to 10° C., or a combination thereof.
1.54. Any one of salts 1.50-1.53, wherein the salt is isolated by filtration from the reaction mixture, optionally wherein the filtration is carried out at 0° C. to 15° C., or 5° C. to 15° C., or 0° C. to 10° C., or 5° C. to 10° C.
1.55. Salt 1.54, wherein after filtration, the salt is washed with solvent (e.g., acetone, 2-butanone, methyl tert-butyl ether, or a combination thereof), optionally washed with cold solvent (e.g., 0° C. to 15° C. or 5° C. to 10° C.) and dried at elevated temperature (e.g., 30-100° C., or 40 to 80° C., or 40 to 60° C., or 40 to 50° C.).
1.56. Any one of salts 1.26-1.55, wherein the salt is white, off-white, pale yellow, light yellow, or gray (e.g., the salt is not purple), optionally wherein the salt does not turn purple.
1.57. Any foregoing form of Salt 1, wherein the salt is an oxalic acid salt prepared from (e.g., crystallized from) acetone, ethyl acetate, acetonitrile, or 2-butanone solvent.
1.58. Any foregoing form of Salt 1, wherein the salt is an oxalic acid salt prepared from (e.g., crystallized from) acetonitrile or ethyl acetate solvent.
1.59. Any foregoing form of Salt 1, wherein the salt is an oxalic acid salt, optionally wherein the salt was prepared in (e.g., crystallized from) acetonitrile solvent, the salt having a DSC/TGA thermogram showing one endothermic event at about 214° C. and a second endothermic event at about 225° C., e.g., wherein the DSC/TGA thermogram shows the endothermic event at about Tₒₙₛₑₜ=209° C., Tₚₑₐₖ=214° C. and ΔE=−88 J/g, for example, wherein the first endothermic event is a melt.
1.60. Any foregoing form of Salt 1, wherein the salt is an oxalic acid salt, optionally wherein the salt was prepared in (e.g., crystallized from) ethyl acetate solvent, the salt having a DSC/TGA thermogram showing one endothermic event at about 215° C., a second endothermic event at about 216° C., and one exothermic event at about 225° C., e.g., wherein the DSC/TGA thermogram shows the first endothermic event at about Tₒₙₛₑₜ=209° C., Tₚₑₐₖ=215° C. and ΔE=−89 J/g, and the second endothermic event at about Tₒₙₛₑₜ=211° C., Tₚₑₐₖ=225° C. and ΔE=−28.8 J/g, for example, wherein the first endothermic event is a desolvation and the second endothermic event is a melt.
1.61. Any foregoing form of Salt 1, wherein the salt is an oxalic acid salt, optionally wherein the salt was prepared in (e.g., crystallized from) acetonitrile solvent or ethyl acetate solvent, the salt having a DSC/TGA thermogram as shown in FIG. 8.
1.62. Any foregoing form of Salt 1, wherein the salt is an oxalic acid salt, optionally wherein the salt was prepared in (e.g., crystallized from) acetonitrile solvent or ethyl acetate solvent, the salt having a crystal structure characterized by an X-ray powder diffraction pattern corresponding to the d-spacing and/or angle (2-theta) values of the following table, for example, wherein the pattern comprises at least five, or at least six, or at least seven, or at least eight of said values, e.g., taking into account potential variations due to sample purity and instrument variation, for example 2θ shifts due to variation in X-ray wavelength, e.g., wherein the X-ray powder diffraction pattern is generated using an X-ray diffractometer with a copper anode and a nickel filter, e.g., comprising at least those peaks having a relative intensity of at least 0.4, at least 0.5, or at least 0.6, or comprising peaks 5, 6, 7, 9, 10, and 12:

XRPD (Cu anode, Ni filter) for Oxalate Salt Crystal Polymorph 1

| # | Angle | d Value | Rel. Intensity |
|---|---|---|---|
| 1 | 12.046 | 7.34112 | 11.90 |
| 2 | 13.515 | 6.54647 | 41.20 |
| 3 | 14.101 | 6.27577 | 15.00 |
| 4 | 15.696 | 5.64131 | 45.40 |
| 5 | 17.203 | 5.1504 | 10.20 |
| 6 | 17.713 | 5.00338 | 22.40 |
| 7 | 18.222 | 4.86465 | 36.70 |
| 8 | 19.833 | 4.47306 | 100.00 |
| 9 | 20.955 | 4.23597 | 65.00 |
| 10 | 22.045 | 4.02886 | 25.70 |
| 11 | 23.2 | 3.83081 | 22.10 |
| 12 | 23.324 | 3.81074 | 24.30 |
| 13 | 24.101 | 3.68958 | 38.30 |
| 14 | 26.153 | 3.40457 | 7.10 |
| 15 | 28.074 | 3.17583 | 19.10 |
| 16 | 29.069 | 3.06939 | 15.90 |
| 22 | 36.548 | 2.45664 | 3.60 |
| 23 | 36.588 | 2.45405 | 4.30 |
| 24 | 36.615 | 2.45229 | 3.80 |

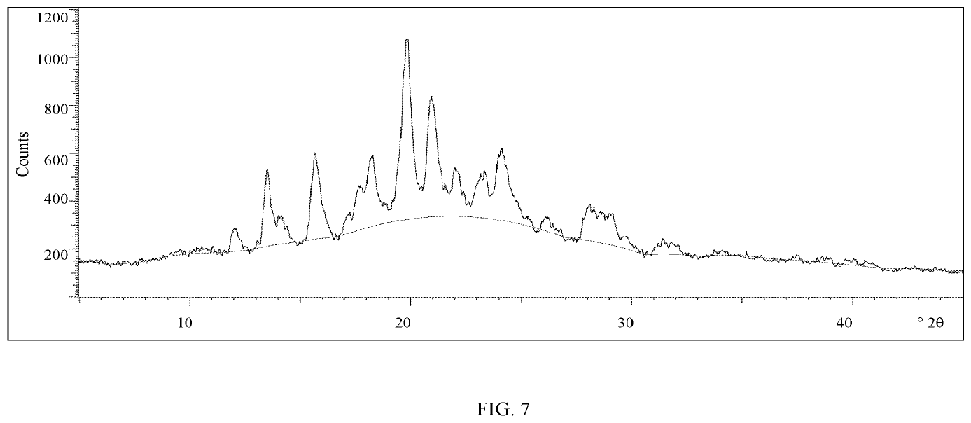

1.63. Any foregoing form of Salt 1, wherein the salt is an oxalic acid salt, optionally wherein the salt was prepared in (e.g., crystallized from) acetonitrile solvent or ethyl acetate solvent, the salt having a crystal structure characterized by an X-ray powder diffraction pattern corresponding to FIG. 7, e.g., taking into account potential variations due to sample purity and instrument variation, for example 2θ shifts due to variation in X-ray wavelength, e.g., an X-ray powder diffraction pattern corresponding to FIG. 1 generated using an X-ray diffractometer with a copper anode and a nickel filter.
1.64. Any foregoing form of Salt 1, wherein the salt is an oxalic acid salt, optionally wherein the salt was prepared in (e.g., crystallized from) acetonitrile solvent or ethyl acetate solvent, the salt having a crystal structure characterized by an X-ray powder diffraction pattern having at least 5, or at least 6, or at least 7, or at least 8, peaks having angle (2-theta) values selected from the group consisting of about 12.1, 13.5, 14.1, 15.7, 17.2, 17.7, 18.2, 19.8, 21.0, 22.1, 23.2, 23.3, 24.1, 26.2, 28.1, and 29.1, taking into account potential variations due to sample purity and instrument variation, e.g., wherein the X-ray powder diffraction pattern is generated using an X-ray diffractometer with a copper anode and a nickel filter.
1.65. Any foregoing form of Salt 1, wherein the salt is an oxalic acid salt, optionally wherein the salt was prepared in (e.g., crystallized from) acetonitrile solvent or ethyl acetate solvent, the salt having a crystal structure characterized by an X-ray powder diffraction pattern having at least 5, or at least 6, or at least 7, or at least 8, peaks having angle (2-theta) values selected from the group consisting of about 12.05, 13.52, 14.10, 15.70, 17.20, 17.71, 18.22, 19.83, 20.96, 22.05, 23.20, 23.32, 24.10, 26.15, 28.07, and 29.07, taking into account potential variations due to sample purity and instrument variation, e.g., wherein the X-ray powder diffraction pattern is generated using an X-ray diffractometer with a copper anode and a nickel filter.
1.66. Any foregoing form of Salt 1, wherein the salt is an oxalic acid salt, optionally wherein the salt was prepared in (e.g., crystallized from) acetonitrile solvent or ethyl acetate solvent, the salt having a crystal structure characterized by an X-ray powder diffraction pattern at least 5, or at least 6, or at least 7, or at least 8, peaks having d-spacing values selected from the group consisting of about 7.34, 6.55, 6.28, 5.64, 5.15, 5.00, 4.86, 4.47, 4.24, 4.03, 3.83, 3.81, 3.69, 3.40, 3.18, and 3.07, taking into account potential variations due to sample purity and instrument variation, wherein the X-ray powder diffraction pattern is generated using an X-ray diffractometer with a copper anode and a nickel filter.
1.67. Any foregoing form of Salt 1, wherein the salt is an oxalic acid salt, optionally wherein the salt was prepared in (e.g., crystallized from) acetonitrile solvent or ethyl acetate solvent, the salt having a crystal structure characterized by an X-ray powder diffraction pattern at least 5, or at least 6, or at least 7, or at least 8, peaks having d-spacing values selected from the group consisting of about 7.3, 6.6, 6.3, 5.6, 5.2, 5.0, 4.9, 4.5, 4.2, 4.0, 3.8, 3.8, 3.7, 3.4, 3.2, and 3.1, taking into account potential variations due to sample purity and instrument variation, wherein the X-ray powder diffraction pattern is generated using an X-ray diffractometer with a copper anode and a nickel filter.
1.68. Any foregoing form of Salt 1, wherein the salt is an oxalic acid salt, optionally wherein the salt was prepared in (e.g., crystallized from) acetonitrile solvent or ethyl acetate solvent, the salt having an X-ray powder diffraction pattern as shown, or substantially as shown, in FIG. 7.
1.69. Any foregoing form of Salt 1, wherein the salt is an oxalic acid salt, optionally wherein the salt was prepared in (e.g., crystallized from) acetonitrile solvent or ethyl acetate solvent, the salt having an X-ray powder diffraction pattern relative angle (2-theta) values as provided in any one of embodiments 1.62 to 1.65 above, wherein the values are shifted by up to +/−0.2 degrees, e.g., wherein the values are substantially uniformly shifted by up to +/−0.2 degrees.
1.70. Any foregoing form of Salt 1 (e.g., a toluenesulfonic acid salt, optionally wherein the salt was prepared in (e.g., crystallized from) 2-butanone solvent or acetone solvent), wherein the salt comprises less than 5000 ppm of all residual solvents (e.g., acetone or 2-butanone), e.g., less than 4000 ppm, or less than 3000 ppm, or less than 2000 ppm, or less than 1000 ppm, or less than 750 ppm, or less than 500 ppm, or less than 250 ppm, or less than 150 ppm, or less than 100 ppm, or less than 50 ppm, of all residual solvents.
1.71. Any foregoing form of Salt 1 (e.g., a toluenesulfonic acid salt, optionally wherein the salt was prepared in (e.g., crystallized from) 2-butanone solvent or acetone solvent), wherein the salt comprises less than 100 ppm of 1-(3-chloropropoxy)-4-fluorobenzene, e.g., less than 75 ppm, or less than 50 ppm, or less than 25 ppm, or less than 20 ppm, or less than 15 ppm, or less than 10 ppm.
1.72. Any foregoing form of Salt 1 (e.g., a toluenesulfonic acid salt, optionally wherein the salt was prepared in (e.g., crystallized from) 2-butanone solvent or acetone solvent), wherein the salt comprises less than 5 wt. % of water, e.g., less than 4 wt. %, or less than 3 wt. %, or less than 2 wt. %, or less than 1 wt. %, or less than 0.9 wt. %, or less than 0.8 wt. %, or less than 0.7 wt. %, or less than 0.6 wt. %, or less than 0.5 wt. % of water.
1.73. Any foregoing form of Salt 1 (e.g., a toluenesulfonic acid salt, optionally wherein the salt was prepared in (e.g., crystallized from) 2-butanone solvent or acetone solvent), wherein the salt comprises less than 100 ppm of p-toluenesulfonate alkyl esters (e.g., methyl, ethyl and/or isopropyl esters), e.g., less than 75 ppm, or less than 50 ppm, or less than 40 ppm, or less than 30 ppm, or less than 20 ppm, of p-toluenesulfonate alkyl esters.
1.74. Any foregoing form of Salt 1 (e.g., a toluenesulfonic acid salt, optionally wherein the salt was prepared in (e.g., crystallized from) 2-butanone solvent or acetone solvent), wherein the salt comprises less than 50 ppm of any p-toluenesulfonate alkyl ester (e.g., methyl, ethyl and/or isopropyl esters), e.g., less than 40 ppm, or less than 30 ppm, or less than 25 ppm, or less than 20 ppm, or less than 15 ppm, or less than 10 ppm, of any p-toluenesulfonate alkyl ester, for example, less than 20 ppm of each p-toluenesulfonate alkyl ester.
1.75. Any foregoing form of Salt 1 (e.g., a toluenesulfonic acid salt, optionally wherein the salt was prepared in (e.g., crystallized from) 2-butanone solvent or acetone solvent), wherein the salt has a particle size of less than 50 microns, e.g., wherein the salt particles have a D90 of less than 50 m, or less than 40 m, or less than 30 m, or less than 20 m, or less than 10 μm.
1.76. Any foregoing form of Salt 1, wherein the salt exhibits any combination of characteristics as described in 1.1-1.75.

In a second embodiment, the invention provides (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one (Compound A) in solid, co-crystal form (Co-Crystal 2).

In additional embodiments, the present disclosure provides the following:
2.1. Co-Crystal 2, wherein the co-crystal is at least 90% pure, e.g., at least 92%, at least 94%, at least 95%, at least 96%, at least 97%, at least 98% or at least 99% pure, for example, as shown by LCMS.
2.2. Co-Crystal 2 or 2.1, wherein the co-crystal comprises less than 10 wt. %, e.g., less than 8 wt. %, or less than 6 wt. %, or less than 5 wt. %, or less than 4 wt. %, or less than 3 wt. %, or less than 2 wt. %, or less than 1 wt. %, or less than 0.5 wt. %, or less than 0.1 wt. %, of Compound A in free base form.
2.3. Co-Crystal 2 or any of 2.1-2.2, wherein the co-crystal comprises less than 10 wt. %, e.g., less than 8 wt. %, or less than 6 wt. %, or less than 5 wt. %, or less than 4 wt. %, or less than 3 wt. %, or less than 2 wt. %, or less than 1 wt. %, or less than 0.5 wt. %, or less than 0.1 wt. %, of Compound A in any other co-crystal form.
2.4. Co-Crystal 2 or any of 2.1-2.3, wherein the salt comprises less than 10 wt. %, e.g., less than 8 wt. %, or less than 6 wt. %, or less than 5 wt. %, or less than 4 wt. %, or less than 3 wt. %, or less than 2 wt. %, or less than 1 wt. %, or less than 0.5 wt. %, or less than 0.1 wt. %, of any free crystal co-former (e.g., alanine, phenylalanine, or another amino acid).
2.5. Co-Crystal 2 or any of 2.1-2.4, wherein the co-crystal comprises less than 10 wt. %, e.g., less than 8 wt. %, or less than 6 wt. %, or less than 5 wt. %, or less than 4 wt. %, or less than 3 wt. %, or less than 2 wt. %, or less than 1 wt. %, or less than 0.5 wt. %, or less than 0.1 wt. %, of any free solvent (e.g., other than solvent comprised in solvated co-crystal forms).
2.6. Co-Crystal 2 or any of 2.1-2.5, wherein the co-crystal comprises less than 10 wt. %, e.g., less than 8 wt. %, or less than 6 wt. %, or less than 5 wt. %, or less than 4 wt. %, or less than 3 wt. %, or less than 2 wt. %, or less than 1 wt. %, or less than 0.5 wt. %, or less than 0.1 wt. %, of any synthetic precursor of, or side-product from, the synthetic method used to prepare the Compound A in free base form.
2.7. Co-Crystal 2 or any of 2.1-2.6, wherein the co-crystal is in a homogeneous crystal form, e.g., free or substantially free of other crystalline forms or polymorphs or amorphous forms of Compound A, e.g., free or substantially free, e.g., less than 10 wt. %, preferably less than about 5 wt. %, more preferably less than about 2 wt. %, still preferably less than about 1 wt. %, still preferably less than about 0.1%, most preferably less than about 0.01 wt. %, of any other crystalline forms or polymorphs or amorphous forms.
2.8. Co-Crystal 2, or any of 2.1-2.7, wherein the co-crystal is a co-crystal with an amino acid (e.g., a natural alpha-amino acid).
2.9. Co-Crystal 2.8, wherein the amino acid is selected from alanine, asparagine, cysteine, methionine, phenylalanine, proline, serine, glutamine, glycine, histidine, lysine, threonine, tyrosine, and valine.
2.10. Co-Crystal 2.8, wherein the amino acid is selected from alanine, cysteine, methionine, phenylalanine, serine, glycine, histidine, and valine.
2.11. Co-Crystal 2.8, wherein the amino acid is alanine.
2.12. Co-Crystal 2.8, wherein the amino acid is phenylalanine.
2.13. Co-Crystal 2, or any of 2.1-2.12, wherein the co-crystal is a co-crystal comprising the Compound A free base and the co-former (e.g., alanine or phenylalanine) in a 1:2, 1:1, 2:1, 3:1, or 4:1 molar ratio.
2.14. Co-Crystal 2, or any of 2.1-2.13, wherein the co-crystal is formed by reacting the Compound A in free base form with the co-former (e.g., alanine or phenylalanine) in a solvent selected from methanol, dimethyl sulfoxide, acetone, or acetonitrile, e.g., acetone or acetonitrile.

In another aspect, the invention provides a process (Process 1) for the production of (6bR,10aS)-8-(3-(4-fluorophenoxy)propyl)-6b,7,8,9,10,10a-hexahydro-1H-pyrido[3′, 4′: 4,5]pyrrolo[1,2,3-de]quinoxalin-2(3H)-one (Compound A) in solid, crystalline salt form (e.g., Salt 1), comprising
(a) reacting (6bR,10aS)-8-(3-(4-fluorophenoxy)propyl)-6b,7,8,9,10,10a-hexahydro-1H-pyrido[3′, 4′: 4,5]pyrrolo[1,2,3-de]quinoxalin-2(3H)-one (Compound A) free base with an acid selected from hydrochloric acid, p-toluenesulfonic acid, tartaric acid, malic acid, fumaric acid, glutamic acid, oxalic acid, benzenesulfonic acid, and ascorbic acid, e.g., together with an organic solvent (e.g., comprising 2-butanone or acetone); and
(b) recovering the salt thus formed, e.g., recovering Salt 1 or any of Salts 1.1-1.76.

In further embodiments of Process 1, the present disclosure provides:
1.1 Process 1, wherein the acid is selected from p-toluenesulfonic acid, tartaric acid, malic acid, fumaric acid, glutamic acid, oxalic acid, benzenesulfonic acid, and ascorbic acid.
1.2 Process 1.1, wherein the acid is selected from p-toluenesulfonic acid and oxalic acid.
1.3 Process 1.2, wherein the acid is oxalic acid and the salt is any one of salts 1.57 to 1.69.
1.4 Process 1.2, wherein the acid is p-toluenesulfonic acid and the salt is any one of salts 1.25 to 1.56 or 1.70 to 1.76.
1.5 Process 1, or any of 1.1 to 1.4, wherein the solvent of step (a) is selected from methanol, dimethyl sulfoxide, acetone, acetonitrile, ethyl acetate, toluene, 2-butanone, benzonitrile, cyclohexane, tetrahydrofuran, and diisopropyl ether, or a mixture thereof, optionally further comprising water or hexane.
1.6 Process 1.5, wherein the solvent is selected from acetone, acetonitrile, ethyl acetate, toluene, and 2-butanone, or a mixture thereof.
1.7 Process 1.6, wherein the solvent is selected from acetone and 2-butanone.
1.8 Process 1.7, wherein the solvent is acetone, e.g., the solvent consists of acetone.
1.9 Process 1 or any of 1.1-1.8, wherein the compound of Formula A and the acid are combined in step (a) in an about 1:0.9 to 1:5 molar ratio, e.g., 1:0.9 to 1:3, or 1:0.9 to 1:1.5, or 1:0.9 to 1:1.2, or 1:0.9 to 1:1.1, or about 1:1, molar ratio.
1.10 Process 1, or any of 1.1-1.9, wherein the in step (a) a solution of the acid in the solvent is add to a solution of the Compound A free base in the solvent.
1.11 Process 1.10, wherein the concentration of the Compound A free base in the solvent (prior to the addition of the acid) is about 0.1 to 0.3 g/mL, e.g., 0.1 to 0.15 g/mL, or about 0.1 or 0.11 g/mL.
1.12 Process 1.10 or 1.11, wherein the concentration of the acid in the solvent (prior to addition of the acid to the free base solution) is about 1M to 5 M, e.g., 2M to 3M, or about 2.5M, for example, wherein the acid is p-toluenesulfonic acid monohydrate and the concentration is about 0.3 to 1 g/mL, e.g., 0.37 to 0.55 g/mL, or about 0.45 g/mL.
1.13 Process 1, or any of 1.1-1.12, wherein step (a) consists of one equivalent of Compound A in free base form, the solvent, and 0.95 to 1.15 equivalents of the acid (e.g., p-toluenesulfonic acid monohydrate), or 0.95 to 1.10 equivalents, or 0.95 to 1.05 equivalents, or 0.95 to 1.00 equivalents, or 0.95 to 0.99 equivalents.
1.14 Process 1.13, wherein the net concentration of the Compound A after addition of all solvent is about 0.1 to 0.3 g/mL, e.g., 0.1 to 0.15 g/mL, or about 0.1 g/mL.
1.15 Process 1, or any of 1.1-1.14, wherein step (a) occurs at a temperature of 0° C. to 40° C., e.g., 0° C. to 30° C., or 0° C. to 25° C., or 0° C. to 15° C., or 5° C. to 20° C., or 5° C. to 15° C., or 10° C. to 20° C., or 10° C. to 15° C., or 5° C. to 10° C., or a combination thereof.
1.16 Process 1.14, wherein the Compound A free base, the acid and the solvent are combined at a temperature of 10° C. to 20° C., or 10° C. to 15° C., and after the combination is complete the reaction is cooled to a temperature of 5° C. to 15° C., or 5° C. to 10° C., or about 5° C.
1.17 Process 1, or any of 1.1-1.16, wherein step (a) occurs for 12-24 hours, e.g., 14-20 hours or 14-16 hours.
1.18 Process 1, or any of 1.1-1.17, wherein step (b) comprises isolating the precipitated salt product by filtration, followed by washing with solvent (e.g., acetone, 2-butanone, methyl tert-butyl ether, or a combination thereof).
1.19 Process 1.18, wherein the filtration and washing are carried out at 0° C. to 15° C., or 5° C. to 15° C., or 0° C. to 10° C., or 5° C. to 10° C.
1.20 Process 1.18 or 1.19, wherein the salt product is washed with cold solvent (e.g., 0° C. to 15° C. or 5° C. to 10° C.).
1.21 Any of Process 1.18-1.20, wherein the washing solvent is acetone.
1.22 Any of Process 1.18-1.21, wherein step (b) further comprises drying the salt product at elevated temperature (e.g., 30-100° C., or 40 to 80° C., or 40 to 60° C., or 40 to 50° C.).
1.23 Process 1, or any of 1.1-1.22, wherein the Process provides a salt comprising less than 5000 ppm of all residual solvents (e.g., any one or more of acetone, methanol, acetonitrile, methyl tert-butyl ether, ethyl acetate, heptane, toluene, dimethylacetamide, diisopropylamine, and dimethylsulfoxide), e.g., less than 4000 ppm, or less than 3000 ppm, or less than 2000 ppm, or less than 1000 ppm, or less than 750 ppm, or less than 500 ppm, or less than 250 ppm, or less than 150 ppm, or less than 100 ppm, or less than 50 ppm, of all residual solvents.
1.24 Process 1, or any of 1.1-1.23, wherein the Process provides a salt comprising less than 5000 ppm of each residual solvent (e.g., any one or more of acetone, methanol, acetonitrile, methyl tert-butyl ether, ethyl acetate, heptane, toluene, dimethylacetamide, diisopropylamine, and dimethylsulfoxide), e.g., less than 4000 ppm, or less than 3000 ppm, or less than 2000 ppm, or less than 1000 ppm, or less than 750 ppm, or less than 500 ppm, or less than 250 ppm, or less than 150 ppm, or less than 100 ppm, or less than 50 ppm, of each residual solvents.
1.25 Process 1, or any of 1.1-1.24, wherein the Process provides a salt comprising less than 100 ppm of 1-(3-chloropropoxy)-4-fluorobenzene, e.g., less than 75 ppm, or less than 50 ppm, or less than 25 ppm, or less than 20 ppm, or less than 15 ppm, or less than 10 ppm.
1.26 Process 1, or any of 1.1-1.25, wherein the Process provides a salt comprising less than 5 wt. % of water, e.g., less than 4 wt. %, or less than 3 wt. %, or less than 2 wt. %, or less than 1 wt. %, or less than 0.9 wt. %, or less than 0.8 wt. %, or less than 0.7 wt. %, or less than 0.6 wt. %, or less than 0.5 wt. % of water.
1.27 Process 1, or any of 1.1-1.26, wherein the Process provides a salt comprising less than 250 ppm of all heavy metals (e.g., arsenic, cadmium, cobalt, lead, nickel, mercury, vanadium, lithium, antimony, and/or copper), e.g., less than 150 ppm, or less than 100 ppm, or less than 50 ppm, or less than 25 ppm, or less than 15 ppm, or less than 10 ppm.
1.28 Process 1, or any of 1.1-1.27, wherein the Process provides a salt comprising less than 50 ppm of any individual heavy metal (e.g., arsenic, cadmium, cobalt, lead, nickel, mercury, vanadium, lithium, antimony, and/or copper), e.g., less than 40 ppm, or less than 30 ppm, or less than 25 ppm, or less than 20 ppm, or less than 15 ppm, or less than 10 ppm, or less than 5 ppm, or less than 1 ppm.
1.29 Process 1.27 or 1.28, wherein the Process provides a salt comprising less than 50 ppm of copper, e.g., less than 40 ppm, or less than 30 ppm, or less than 25 ppm, or less than 20 ppm, or less than 15 ppm, or less than 10 ppm, or less than 5 ppm, or less than 1 ppm.
1.30 Process 1, or any of 1.1-1.29, wherein the Process provides a salt having a particle size of less than 50 microns, e.g., wherein the salt particles have a D90 of less than 50 m, or less than 40 m, or less than 30 m, or less than 20 m, or less than 10 am.
1.31 Process 1, or any of 1.1-1.30, wherein the Process provides a p-toluenesulfonic acid salt of Compound A, e.g., a monotosylate salt.
1.32 Process 1.31, wherein the salt is formed as a white, off-white, pale yellow, light yellow, or gray solid (e.g., the salt is not purple).
1.33 Process 1.31 or 1.32, wherein the Process provides a salt comprising less than 100 ppm of p-toluenesulfonate alkyl esters (e.g., methyl, ethyl and/or isopropyl esters), e.g., less than 75 ppm, or less than 50 ppm, or less than 40 ppm, or less than 30 ppm, or less than 20 ppm, of p-toluenesulfonate alkyl esters.
1.34 Process 1.31, 1.32, or 1.33, wherein the Process provides a salt comprising less than 50 ppm of any p-toluenesulfonate alkyl ester (e.g., methyl, ethyl and/or isopropyl esters), e.g., less than 40 ppm, or less than 30 ppm, or less than 25 ppm, or less than 20 ppm, or less than 15 ppm, or less than 10 ppm, of any p-toluenesulfonate alkyl ester, for example, less than 20 ppm of each p-toluenesulfonate alkyl ester
1.35 Any of Process 1.31-1.34, wherein the solvent of step (a) is acetone and wherein the washing solvent of step (b) is acetone.
1.36 Process 1, or any of 1.1-1.35, wherein in step (a) the Compound A in free base form is recrystallized Compound A in free base form, e.g., recrystallized from an acetone or acetone/methanol solution or suspension.

In another aspect, the invention provides a process (Process 2) for the production of Co-Crystal 2, comprising
(a) reacting (6bR,10aS)-8-(3-(4-fluorophenoxy)propyl)-6b,7,8,9,10,10a-hexahydro-1H-pyrido[3′, 4′: 4,5]pyrrolo[1,2,3-de]quinoxalin-2(3H)-one (Compound A) free base with a crystal co-former, optionally selected from an amino acid selected from alanine, asparagine, cysteine, methionine, phenylalanine, proline, serine, glutamine, glycine, histidine, lysine, threonine, tyrosine, and valine (e.g., alanine or phenylalanine), for example, together with an organic solvent (e.g., selected from methanol, dimethyl sulfoxide, acetone, or acetonitrile, e.g., acetone or acetonitrile); and
(b) recovering the co-crystal thus formed, e.g., recovering Co-Crystal 2 or any of 2.1-2.14.

In another embodiment, the invention provides a pharmaceutical composition comprising Salt 1, or any of Salts 1.1-1.76, or Co-Crystal 2 or any of 2.1-2.14, as active ingredient, in combination or association with a pharmaceutically acceptable diluent or carrier.

In another embodiment, the invention provides a pharmaceutical composition comprising Salt 1, or any of Salts 1.1-1.76, or Co-Crystal 2 or any of 2.1-2.14, as active ingredient, in combination or association with a pharmaceutically acceptable diluent or carrier, wherein the salt is predominantly, or is entirely or substantially entirely, in dry crystalline form.

In a particular embodiment, the invention provides a pharmaceutical composition comprising Salt 1, or any of Salts 1.1-1.76, or Co-Crystal 2 or any of 2.1-2.14, as active ingredient, in combination or association with a pharmaceutically acceptable diluent or carrier, wherein the composition is in the form of an injectable depot, e.g., to provide extended release of Compound A.

In some embodiments, the Pharmaceutical Composition is selected from a tablet, capsule, caplet, powder, wafer, gel, or sterile injectable solution. In some embodiments, the Pharmaceutical Composition is an orally disintegrating tablet. In some embodiments, the Pharmaceutical Composition is a long-acting injectable composition, e.g., for intramuscular or subcutaneous administration. In some embodiments, the Pharmaceutical Composition comprises from 1 to 60 mg of the Compound A, measured by weight of the equivalent free base (e.g., from 20-60 mg, or 20-40 mg, or 40-60 mg, for an oral ingested dosage form; e.g., from 1-30 mg, or 5-20 mg, or 5-15 mg, or 1-10 mg, for an oral rapidly dissolving dosage form).

In another embodiment, the invention provides Salt 1, or any of Salts 1.1-1.76, or Co-Crystal 2 or any of 2.1-2.14, or a pharmaceutical composition comprising Salt 1, or any of Salts 1.1-1.76, or Co-Crystal 2 or any of 2.1-2.14, for use in treating a disease or abnormal condition involving or mediated by the 5-HT_{2A} receptor, serotonin transporter (SERT), dopamine D₁/D₂ receptor signaling pathways, and/or the mu-opioid receptor, e.g., a disorder selected from obesity, anxiety, depression (for example refractory depression and MDD), psychosis (including psychosis associated with dementia, such as hallucinations in advanced Parkinson's disease or paranoid delusions), schizophrenia, sleep disorders (particularly sleep disorders associated with schizophrenia and other psychiatric and neurological diseases), sexual disorders, migraine, conditions associated with cephalic pain, social phobias, agitation in dementia (e.g., agitation in Alzheimer's disease), agitation in autism and related autistic disorders, gastrointestinal disorders such as dysfunction of the gastrointestinal tract motility, and dementia, for example dementia of Alzheimer's disease or of Parkinson's disease; mood disorders; and drug dependencies, for example, opiate or opioid dependency and/or alcohol dependency, or withdrawal from drug or alcohol dependency (e.g., opiate or opioid dependency); or binge eating disorder; or opioid overdose; or opioid use disorder (OUD); or substance use disorder or substance abuse disorder (e.g., as these terms are defined in the DSM-V), optionally in patient also suffering from anxiety and/or depression; or obsessive-compulsive disorder (OCD), obsessive-compulsive personality disorder (OCPD), general anxiety disorder, social anxiety disorder, panic disorder, agoraphobia, compulsive gambling disorder, compulsive eating disorder, body dysmorphic disorder, hypochondriasis, pathological grooming disorder, kleptomania, pyromania, attention deficit-hyperactivity disorder (ADHD), attention deficit disorder (ADD), impulse control disorder, and related disorders, and combinations thereof; or a pain disorder, e.g., a condition associated with pain, such as cephalic pain, idiopathic pain, neuropathic pain, chronic pain (e.g., moderate to moderately severe chronic pain, for example, in patients requiring 24-hour extended treatment for other ailments), fibromyalgia, dental pain, traumatic pain, or chronic fatigue; or wherein the central nervous system disease or disorder is a drug dependency (for example, opiate or opioid dependency (i.e., opioid use disorder), cocaine dependency, amphetamine dependency, and/or alcohol dependency), or withdrawal from drug or alcohol dependency (e.g., opiate, opioid, cocaine, or amphetamine dependency), and wherein the patient also suffers from a co-morbidity, such as anxiety, depression or psychosis, and/or wherein the patient also suffers from an opiate or opioid overdose.

In another embodiment, the invention provides a method for the prophylaxis or treatment of a human suffering from a disease or abnormal condition involving or mediated by the 5-HT_{2A} receptor, serotonin transporter (SERT), dopamine D₁/D₂ receptor signaling pathways, and/or the mu-opioid receptor, e.g., a disorder selected from obesity, anxiety, depression (for example refractory depression and MDD), psychosis (including psychosis associated with dementia, such as hallucinations in advanced Parkinson's disease or paranoid delusions), schizophrenia, sleep disorders (particularly sleep disorders associated with schizophrenia and other psychiatric and neurological diseases), sexual disorders, migraine, conditions associated with cephalic pain, social phobias, agitation in dementia (e.g., agitation in Alzheimer's disease), agitation in autism and related autistic disorders, gastrointestinal disorders such as dysfunction of the gastrointestinal tract motility, and dementia, for example dementia of Alzheimer's disease or of Parkinson's disease; mood disorders; and drug dependencies, for example, opiate or opioid dependency and/or alcohol dependency, or withdrawal from drug or alcohol dependency (e.g., opiate or opioid dependency); or binge eating disorder; or opiate or opioid overdose; or opioid use disorder (OUD), or substance use disorder or substance abuse disorder (e.g., as these terms are defined in the DSM-V), optionally in patient also suffering from anxiety and/or depression; or obsessive-compulsive disorder (OCD), obsessive-compulsive personality disorder (OCPD), general anxiety disorder, social anxiety disorder, panic disorder, agoraphobia, compulsive gambling disorder, compulsive eating disorder, body dysmorphic disorder, hypochondriasis, pathological grooming disorder, kleptomania, pyromania, attention deficit-hyperactivity disorder (ADHD), attention deficit disorder (ADD), impulse control disorder, and related disorders, and combinations thereof; or a pain disorder, e.g., a condition associated with pain, such as cephalic pain, idiopathic pain, neuropathic pain, chronic pain (e.g., moderate to moderately severe chronic pain, for example, in patients requiring 24-hour extended treatment for other ailments), fibromyalgia, dental pain, traumatic pain, or chronic fatigue; or wherein the central nervous system disease or disorder is a drug dependency (for example, opiate or opioid dependency (i.e., opioid use disorder), cocaine dependency, amphetamine dependency, and/or alcohol dependency), or withdrawal from drug or alcohol dependency (e.g., opiate, opioid, cocaine, or amphetamine dependency), and wherein the patient also suffers from a co-morbidity, such as anxiety, depression or psychosis, and/or wherein the patient also suffers from an opiate or opioid overdose; the method comprising administering to a patient in need thereof a therapeutically effective amount of Salt 1, or any of Salts 1.1-1.76, or Co-Crystal 2 or any of 2.1-2.14.

In some embodiments of the preceding methods and uses, the patient is not responsive to or cannot tolerate the side effects from one or more of: treatment with selective serotonin reuptake inhibitors (SSRIs), such as citalopram, escitalopram, fluoxetine, fluvoxamine, paroxetine, and sertraline; treatment with serotonin-norepinephrine reuptake inhibitors (SNRIs), such as venlafaxine, sibutramine, duloxetine, atomoxetine, desvenlafaxine, milnacipran, and levomilnacipran; treatment with antipsychotic agents, such as clomipramine, risperidone, quetiapine and olanzapine; treatment with non-narcotic analgesics and/or opiate and opioid drugs, or wherein the use of opiate drugs are contraindicated in said patient, for example, due to prior substance abuse or a high potential for substance abuse, such as opiate and opioid drugs including, e.g., morphine, codeine, thebaine, oripavine, morphine dipropionate, morphine dinicotinate, dihydrocodeine, buprenorphine, etorphine, hydrocodone, hydromorphone, oxycodone, oxymorphone, fentanyl, alpha-methylfentanyl, alfentanyl, trefantinil, brifentanil, remifentanil, ocfentanil, sufentanil, carfentanyl, meperidine, prodine, promedol, propoxyphene, dextropropoxyphene, methadone, diphenoxylate, dezocine, pentazocine, phenazocine, butorphanol, nalbuphine, levorphanol, levomethorphan, tramadol, tapentadol, and anileridine, or any combinations thereof.

In some embodiments of the preceding methods and uses, the patient suffers from a gastrointestinal disorder and/or a pulmonary disorder. Traditional opioid analgesics suffer from two dominant side effects: gastrointestinal disturbances (including nausea, vomiting and constipation) and respiratory depression. 90 to 95% of patients taking opioids for long-term pain treatment develop serious constipation, requiring the long-term use of laxatives and/or enemas.

The stronger opioids such as morphine, oxycodone and hydromorphone produce more severe constipation than other opioids. Respiratory depression is the most serious adverse effect of opioid treatment as it creates a risk of death, especially when patients combine (intentionally or inadvertently) prescribed opioid analgesics with other licit or illicit respiratory depressants (including alcohol). Patients in need of pain treatment, especially chronic pain treatment, are therefore at particular risk of adverse effects if they suffer from a pre-existing gastrointestinal or pulmonary disorder. Unlike traditional opioid analgesics, the compounds of the present invention (e.g., Salt 1, or any of Salts 1.1-1.76, or Co-Crystal 2 or any of 2.1-2.14), provide analgesic relief without significant adverse gastrointestinal effects and without significant respiratory depression. Therefore, such compounds would provide improved safety and efficacy for patients in need of pain treatment having these preexisting GI and pulmonary disorders. In further embodiments, a compound of the present invention may be combined with a traditional opiate agent to provide improved pain control with a dose-sparing effect as to the traditional opiate agent (and concomitantly reduced risk of adverse effects).

This in some embodiments of the methods and uses described above, the patient suffers from a pre-existing or co-morbid gastrointestinal disorder and/or pulmonary disorder, for example, wherein the pre-existing or co-morbid disorder is selected from the group consisting of irritable bowel syndrome, pelvic floor disorder, diverticulitis, inflammatory bowel disease, colon or colorectal cancer, celiac disease, non-celiac gluten sensitivity, asthma, chronic obstructive pulmonary disease (COPD), dyspnea, pneumonia, congestive heart failure, interstitial lung disease, pneumothorax, bronchitis, pulmonary embolism, and traumatic chest injury (e.g., broken sternum or ribs, bruised intercostal muscles). In some embodiments, the central nervous system disorder is a pain disorder, e.g., a condition associated with pain, such as cephalic pain, idiopathic pain, neuropathic pain, chronic pain (e.g., moderate to moderately severe chronic pain, for example, in patients requiring 24-hour extended treatment for other ailments), fibromyalgia, dental pain, traumatic pain, or chronic fatigue. In some embodiments, the central nervous system disorder is opioid use disorder, opiate or opioid withdrawal, or opiate or opioid dependency, and the method provides relief from withdrawal-induced symptoms (e.g., gastrointestinal symptoms such as diarrhea, anxiety, depression, pain, sleep disturbances, or any combination thereof).

In some embodiments of the preceding methods and uses, the method further comprises the concurrent administration of another opiate or opioid agent, e.g., administered simultaneously, separately or sequentially, for example, wherein the additional opiate or opioid agent is selected from the group consisting of morphine, codeine, thebaine, oripavine, morphine dipropionate, morphine dinicotinate, dihydrocodeine, buprenorphine, etorphine, hydrocodone, hydromorphone, oxycodone, oxymorphone, fentanyl, alpha-methylfentanyl, alfentanyl, trefantinil, brifentanil, remifentanil, ocfentanil, sufentanil, carfentanyl, meperidine, prodine, promedol, propoxyphene, dextropropoxyphene, methadone, diphenoxylate, dezocine, pentazocine, phenazocine, butorphanol, nalbuphine, levorphanol, levomethorphan, tramadol, tapentadol, and anileridine, or any combinations thereof. Furthermore, in some embodiments of the preceding methods and uses, the method further comprises the concurrent administration of one or more therapeutic agents selected from the foregoing and further selected from agonists or partial agonists, or inverse agonists or antagonists, of the mu-opioid, kappa-opioid, delta-opioid, and/or nociceptin/orphanin receptors, e.g., an opioid receptor antagonist or inverse agonist, such as a full opiate antagonist, for example, selected from naloxone, naltrexone, nalmefene, methadone, nalorphine, levallorphan, samidorphan, nalodeine, cyprodime, or norbinaltorphimine.

Procedures for the production of Compound A and similar compounds, and synthetic intermediates useful therefor, are known to those skilled in the art, and can be found, for example, in U.S. Pat. Nos. 10,245,260, 10,799,500, 10,961,245, 10,906,906; WO 2020/131895, and WO 2020/131911; the contents of each of which are hereby incorporated by reference in their entireties.

### Examples

The following equipment and methods are used to isolate and characterize the exemplified salt forms:

X-ray powder diffraction (XRPD): The X-ray powder diffraction studies are performed using a Bruker AXS D2 PHASER in Bragg-Brentano configuration, equipment #1549. The equipment uses a Cu anode at 30 kV, 10 mA; sample stage standard rotating; monochromatization by a Kβ-filter (0.5% Ni). Slits: fixed divergence slits 1.0 mm (=0.61°), primary axial Soller slit 2.5°, secondary axial Soller slit 2.5°. Detector: Linear detector LYNXEYE with receiving slit 5° detector opening. The standard sample holder (0.1 mm cavity in (510) silicon wafer) has a minimal contribution to the background signal. Measurement conditions: scan range 5-45° 2θ, sample rotation 5 rpm, 0.5 s/step, 0.010°/step, 3.0 mm detector slit; and all measuring conditions are logged in the instrument control file. The software used for data collection is Diffrac.Commander v4.6. Data analysis is performed using Diffrac.Eva v4.1.1 software. No background correction or smoothing is applied to the patterns.

Simultaneous thermogravimetry (TGA) and differential scanning calorimetry (TGA/DSC) analysis: The TGA/DSC studies are performed using a Mettler Toledo TGA/DSC1 STARe System with a 34-position autosampler, using pierced aluminum crucibles of 40 μl. Typically 5-10 mg of sample is loaded into the crucible and is kept at 20° C. for 5 minutes, after which it is heated at 10° C./min from 20° C. to 350° C. A nitrogen purge of 40 ml/min is maintained over the sample. The software used for instrument control and data analysis is STARe v15.00. No corrections are applied to the thermogram.

Separate differential scanning calorimetry analysis (DSC): The DSC study is performed using a Mettler Toledo HP DSC1 with camera, using an open aluminum standard pan of 40 μl. The sample is heated at 4° C./min from 25° C. to 350° C. under 1 bar of nitrogen. Separate thermogravimetric (TG) analysis: The TGA is performed using a Mettler Toledo TGA/SDTA851e, using an open aluminum standard pan of 40 μl. The sample is heated at 4° C./min from 25° C. to 350° C. under 1 bar of nitrogen.

Fourier transform infrared spectroscopy (FT-IR): The FT-IR studies are performed using a Thermo Scientific Nicolet iS50, equipment #2357. An attenuated total reflectance (ATR) technique is used with a beam splitter of KBr. Number of scans is 16 with a resolution of 4.000, from 400 cm⁻¹ to 4000 cm⁻¹. The software OMNIC version 9.2 is used for data collection and evaluation.

High performance liquid chromatography (HPLC): The high-performance liquid chromatography analyses are performed on an Agilent 1290 system, including CSH C18 column (50 mm×2.1 mm; 1.7 m particle size), degasser, pump, autosampler, thermostat, and DAD-type detector operating at 230 nm. The column is run at a flow rate of 1 mL/min, at 35° C., for a 12-minute run time as follows: 2 minute gradient from A/B 98:2 to 75:25; 6 minute gradient from A/B 75:25 to 50:50; 2 minute gradient from A/B 50:50 to 10:90; 2 minutes at A/B 98:2. Mobile Phase A: Milli-Q water with 0.1% formic acid; Mobile Phase B: acetonitrile with 0.1% formic acid. Sample diluent is 50/50 methanol/acetonitrile and injection volume is 2.0 μl.

Proton Nuclear Magnetic Resonance (NMR): Samples are prepared in DMSO-d6 solvent, and spectra are collected on an Agilent Inova400 at room temperature, and at a frequency of 399.9 MHz, with a sweep width of 6398 Hz, and spin of 20 Hz.

(6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one (Compound A) was synthesized and recrystallized as described in WO 2020/131895. Compound A is a brown crystalline powder with a melting event at a peak temperature of 145° C. The compound exists as agglomerated blocks of less than 20 μm size and it is slightly hygroscopic. The compound is freely soluble in only in DMSO. It is sparingly soluble in ethyl salicylate and anisole. It is less than sparingly soluble (<11 mg/mL) in 2-butanol, methanol, water, ethyl acetate, heptane, and cyclohexane.

### Example 1: Initial Salt Screening

A first initial salt screening is performed by using six different solvents (methanol, DMSO, acetone, acetonitrile, ethyl acetate, toluene) and 14 different acids. Each experiment is conducted at a 1:1 molar ratio of Compound A free base to acid using 30 mg of the free base and 800 μL of solvent, except that two acids (hydrochloric acid and sulfuric acid) are tested at both 1:1 and 1:3 molar ratios. The salt screening, including slurry experiments, cooling crystallization, anti-solvent, shake slurry experiments, are conducted on the Freeslate CM2 equipment.

30 mg of Compound A free base is dispensed into each well of a master plate and a shake slurry plate (both 96 well plate format). Solid acids were added, followed by the solvent, or the solvent was added first followed by liquid acids. The master plate is then heated to 50° C., and is allowed to equilibrate at this temperature for 2 hours. Each well is equipped with a stirring bar (stirring speed is 500 rpm). Two hours later, the residue is filtered, and the filtrate is transferred to the cooling crystallization plate or the precipitation plate.

The cooling crystallization plate is cooled slowly from 50° C. to 10° C., over 8 hours, using an inversed cubic rate. The precipitation plate is filled with water or heptane (300 μl) as anti-solvent (depending on the miscibility with the screening solvents. The shake slurry plate is shaken for 24 hours at room temperature. All obtained solids are characterized by XRPD.

384 combinations of acid, solvent, and method are tested. The vast majority result in either no solid formation, amorphous solid, or solid identified as Compound A free base or acid. 31 reaction conditions produce crystalline solid that is neither Compound A free base nor acid, and XRPD of these 31 solids demonstrates 20 distinct patterns.

These presumptive salt crystal patterns are obtained using hydrochloric acid, p-toluenesulfonic acid, L-tartaric acid, L-ascorbic acid, fumaric acid, sulfuric acid, acetic acid, succinic acid, L-malic acid, glutamic acid, and citric acid. No XRPD patterns are obtained for experiments using phosphoric acid, benzoic acid, or maleic acid. Successful crystallizations, for the most part, only occur using acetone, acetonitrile, ethyl acetate, or toluene solvent. Most of the positive results are obtained using the slurry or the shake slurry methods.

The successful experiments were repeated at a somewhat larger scale (50 mg) in order to have sufficient material for characterization and salt confirmation. Unexpectedly, only hydrochloric acid, p-toluenesulfonic acid, L-tartaric acid, and L-ascorbic acid generated XRPD crystalline salt products on the larger scale, and the results varied depending on the solvent and method used:

| Solvent | Acid | Ratio | Method | Appearance | XRPD |
|---|---|---|---|---|---|
| Toluene | HCl (IPA) | 1:3 | Slurry | Purple solid | Crystalline |
| DMSO | Sulfuric acid | 1:3 | Slurry | Purple solution | Amorphous |
| Acetone | p-toluenesulfonic acid | 1:1 | Slurry | Brown sticky solid | Crystalline |
| Acetonitrile | p-toluenesulfonic acid | 1:1 | Slurry | Purple solid | Amorphous |
| Ethyl acetate | p-toluenesulfonic acid | 1:1 | Slurry | Purple solid | Crystalline |
| Toluene | p-toluenesulfonic acid | 1:1 | Slurry | Purple solid | Crystalline |
| Acetone | Succinic acid | 1:1 | Slurry | Brown sticky solid | Amorphous |
| Acetonitrile | Succinic acid | 1:1 | Slurry | Brown sticky solid | Amorphous |
| Ethyl acetate | Succinic acid | 1:1 | Slurry | Brown sticky solid | Amorphous |
| Acetonitrile | L-tartaric acid | 1:1 | Slurry | Brown sticky solid | Amorphous |
| Ethyl acetate | L-tartaric acid | 1:1 | Slurry | Brown solid (low yield) | Crystalline |
| Acetone | L-ascorbic acid | 1:1 | Slurry | Brown sticky solid | N/A |
| Ethyl acetate | L-ascorbic acid | 1:1 | Slurry | Brown solid | Crystalline |
| Acetone | Fumaric acid | 1:1 | Slurry | Brown solution | N/A |
| Acetone | Succinic acid | 1:1 | Cooling | Brown solid | Amorphous |
| Tol/heptane | Sulfuric acid | 1:3 | Precip. | No precipitation | N/A |
| Tol/heptane | Acetic acid | 1:1 | Precip. | Oil | N/A |
| Ace/water | L-malic acid | 1:1 | Precip. | No precipitation | N/A |
| Tol/heptane | L-malic acid | 1:1 | Precip. | Brown solid | Free base |
| Methanol | HCl (IPA) | 1:1 | Shake | Purple solution | Amorphous |
| Acetone | HCl (IPA) | 1:3 | Shake | Brown solid | Crystalline |
| Acetonitrile | HCl (IPA) | 1:3 | Shake | Purple solid | Crystalline |
| Acetone | p-toluenesulfonic acid | 1:1 | Shake | Brown sticky solid | N/A |
| Acetonitrile | p-toluenesulfonic acid | 1:1 | Shake | Purple solid | Amorphous |
| Ethyl acetate | p-toluenesulfonic acid | 1:1 | Shake | Purple solid | Crystalline |
| Ethyl acetate | Succinic acid | 1:1 | Shake | Brown sticky solid | Poor cryst. |
| Methanol | L-ascorbic acid | 1:1 | Shake | Brown solid | N/A |
| Acetone | L-ascorbic acid | 1:1 | Shake | Brown solid | Poor cryst. |
| DMSO | L-malic acid | 1:1 | Shake | Brown solution | N/A |
| Acetone | Glutamic acid | 1:1 | Shake | Off-white solid | Acid |
| Methanol | Citric acid | 1:1 | Shake | Brown solid | Amorphous |
| DMSO | Citric acid | 1:1 | Shake | Brown solution | N/A |

(Slurry: 50° C. slurry method; Cooling: cooling crystallization method; Precip.: precipitation method; Shake: shake slurry method).

A second, third and fourth initial salt screening experiments are performed with the same solvents and a diverse set of acids (44 additional acids are tested, for a total of 768 additional reaction conditions). Several new crystalline XRPD patterns are obtained at the initial screening scale (30 mg), and successful results are repeated at 50 mg for further characterization of the products. At the larger scale, only galacteric acid, oxalic acid, thiocyanic acid, orotic acid, and gentisic acid, yield crystalline salts. However, ¹H-NMR analysis fails to confirm the crystalline solid as salts of Compound A.

### Example 2: Further Salt Screening Development

Further experiments are performed using some of the successful acid/solvent/method conditions from Example 1, with variations in the solvent volume and/or material concentrations and/or changes to the molar ratio of Compound A free base to acid (1:1.2, 1:1.5 or 1:2). Some experiments are further repeated at a 500 mg scale for confirmation of the results, including additional analysis, including 1H-NMR, DSC/TGA, and/or FTIR.

It is found that the hydrochloride crystals show decomposition by NMR. Confirmed Compound A crystalline salts are observed for p-toluenesulfonic acid (1:1, 1:1.5, or 1:2 molar ratio using ethyl acetate or toluene solvent), L-ascorbic acid (1:1 or 1:2 molar ratio using ethyl acetate or acetone solvent), L-tartaric acid (1:1.5 or 1:3 molar ratio, using ethyl acetate solvent), succinic acid (1:2 molar ratio using ethyl acetate solvent). At a 500 mg scale, L-tartaric acid and L-ascorbic acid reliably produce a crystalline salt using a 1:2 molar ratio of free base to acid (ethyl acetate or acetone solvent, respectively).

An additional set of experiments is performed using a set of 8 acids (acetic, citric, fumaric, L-ascorbic, L-aspartic, L-malic, L-tartaric, succinic) and six solvents (toluene, methanol, 2-butanone, benzonitrile, cyclohexane, diisopropyl ether) using a room temperature slurry method. Positive results are obtained only for fumaric acid/methanol, L-malic acid/methanol, and L-tartaric acid/cyclohexane. The fumarate is a brown powder, the maleate is a dark brown sticky solid and the tartrate is a light-brown powder. Further experiments at a larger scale using fumaric acid, ascorbic acid, and tartaric acid produce reliable salt crystals only for tartaric acid, with best results using ethyl acetate solvent.

Further screening experiments are then performed using tartaric acid, ascorbic acid, fumaric acid, malic acid, hydrochloric acid, p-toluenesulfonic acid, oxalic acid, and benzenesulfonic acid, in additional solvents (acetonitrile, ethyl acetate, propionitrile, benzonitrile, anisole, butyl acetate, ethyl formate, tetrahydrofuran, cyclopentyl methyl ether, di-butyl ether, acetone, methanol, ethanol, 1-propanol, 2-propanol, 2-butanol, 2-butanone, DMSO, toluene). Each of these experiments is performed at a 50 mg scale using 1.5 equivalents of the acid and 300 μL of solvent. The following positive results are obtained:

| Counter ion | Solvent | Experiment | Appearance | XRPD |
|---|---|---|---|---|
| Tartaric acid | Ethyl formate | Slurry 50° C. | Brown sticky | Crystalline |
| Ascorbic acid | Acetone | Slurry 50° C. | Dark brown sticky | Crystalline |
| Malic acid | Toluene | Slurry 50° C. | Brown solid | Crystalline |
| p-Toluenesulfonic acid | Acetone | Slurry 50° C. | Brown powder | Crystalline |
| p-Toluenesulfonic acid | Ethyl acetate | Slurry 50° C. | Purple powder | Crystalline |
| p-Toluenesulfonic acid | 2-Butanone | Slurry 50° C. | Brown powder | Crystalline |
| p-Toluenesulfonic acid | Acetone | Slurry RT | Brown powder | Crystalline |
| p-Toluenesulfonic acid | Acetonitrile | Slurry RT | Purple sticky solid | Crystalline |
| Oxalic acid | Acetone | Slurry 50° C. | Green powder | Crystalline |
| Oxalic acid | Acetonitrile | Slurry 50° C. | Purple powder | Crystalline |
| Oxalic acid | Ethyl acetate | Slurry 50° C. | Purple powder | Crystalline |
| Oxalic acid | Toluene | Slurry 50° C. | Brown powder | Crystalline |
| Oxalic acid | 2-Butanone | Slurry 50° C. | Green powder | Crystalline |
| Oxalic acid | THF | Slurry 50° C. | Purple powder | Crystalline |
| Oxalic acid | Ethyl formate | Slurry 50° C. | Purple sticky solid | Crystalline |

Further experiments are performed at a 200 mg scale with 1.2 mL of solvent, using p-toluenesulfonic acid and oxalic acid. Successful results are obtained using acetone, 2-butanone for p-toluenesulfonic acid, and using acetone, acetonitrile, ethyl acetate, toluene, or 2-butanone for oxalic acid.

Finally, these experiments are repeated at 500 mg scale, with the following results.

| Exp | Counter ion | Solvent | Experiment | Appearance | XRPD |
|---|---|---|---|---|---|
| 2-A | p-Toluenesulfonic acid | 2-Butanone | Slurry 50° C. | Brown powder | Crystalline |
| 2B | p-Toluenesulfonic acid | Acetone | Slurry RT | Brown powder | Crystalline |
| 2-C | Oxalic acid | Acetone | Slurry 50° C. | Green clay | Crystalline |
| 2-D | Oxalic acid | Acetonitrile | Slurry 50° C. | Purple powder | Crystalline |
| 2-E | Oxalic acid | Ethyl acetate | Slurry 50° C. | Purple powder | Crystalline |
| 2-F | Oxalic acid | 2-Butanone | Slurry 50° C. | Green clay | Crystalline |
| 2-G | Oxalic acid | THF | Slurry 50° C. | Purple clay | Crystalline |

XRPD overlays demonstrate that the same p-toluenesulfonic acid salt polymorph forms using both 2-butanone and acetone solvent. The XRPD pattern observed for experiments 2-A and 2-B3 is the same as that shown in FIG. 1 (from Example 3). ¹H-NMR spectra are also substantially the same and demonstrate the formation of a 1:1 free base/acid salt. LCMS shows a purity of about 92% for salt 2-A and 88% for salt 2-B3. FTIR spectra are also substantially the same for both products and consistent with formation of a salt.

TGA/DSC shows that the two p-toluenesulfonic acid salt crystals have thermograms showing similar events, although peak shape and onset temperatures are slightly different. This likely is indicative of differences in solvent trapping in the crystal structures. An overlay of the TGA/DSC thermogram for the p-toluenesulfonic acid salt crystals 2-B (top) and 2-A (bottom) is shown in FIG. 2.

XRPD overlays demonstrate that the same oxalic acid salt polymorph forms using each of the tested solvents (Exps. 2-C to 2-G). The product from Experiments 2-E and 2-F are selected for further analysis. ¹H-NMR spectra shows that the products 2-E and 2-F are substantially the same and demonstrate the formation of a 1:1 free base/acid salt. LCMS shows a purity of about 88% for salt 2-E and 84% for salt 2-F. FTIR spectra are also substantially the same for both products and consistent with formation of a salt.

The XRPD pattern for Experiment 2-E is shown in FIG. 7. The XRPD Peak List for FIG. 7 is shown below:

| | | | Rel. |
|---|---|---|---|
| | Angle | d Value | Intensity |
| Index | (° 2Theta) | (Å) | (%) |
| 1 | 12.046 | 7.34112 | 11.90 |
| 2 | 13.515 | 6.54647 | 41.20 |
| 3 | 14.101 | 6.27577 | 15.00 |
| 4 | 15.696 | 5.64131 | 45.40 |
| 5 | 17.203 | 5.1504 | 10.20 |
| 6 | 17.713 | 5.00338 | 22.40 |
| 7 | 18.222 | 4.86465 | 36.70 |
| 8 | 19.833 | 4.47306 | 100.00 |
| 9 | 20.955 | 4.23597 | 65.00 |
| 10 | 22.045 | 4.02886 | 25.70 |
| 11 | 23.2 | 3.83081 | 22.10 |
| 12 | 23.324 | 3.81074 | 24.30 |
| 13 | 24.101 | 3.68958 | 38.30 |
| 14 | 26.153 | 3.40457 | 7.10 |
| 15 | 28.074 | 3.17583 | 19.10 |
| 16 | 29.069 | 3.06939 | 15.90 |

TGA/DSC shows that the two oxalic acid salt crystals 2-E and 2-F have thermograms showing similar events, although peak shape and onset temperatures are slightly different. This likely is indicative of differences in solvent trapping in the crystal structures. An overlay of the TGA/DSC thermogram for the oxalic acid salt crystals 2-F (top) and 2-E (bottom) is shown in FIG. 8. The mass loss observed in the temperature range of 160° C. to 240° C. (˜25 wt.-%) may be decomposition of the oxalate salt.

These results demonstrate the reproducibility of production of Compound A p-toluenesulfonic acid and oxalate crystal salts.

The tosylate salt shows an improved solubility (1.2-1.7 mg/mL in water) compared to the oxalate salt (0.2-0.3 mg/mL in water).

### Example 3: Toluenesulfonate Salt Scale-Up

(6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one in free base form (1.88 g) is added to a 20 mL vial. 11 mL of 2-butanone is added, and the reaction mixture is heated to 50° C., forming a brown suspension. Solid toluenesulfonic acid (1.5 eq) is added and the mixture soon becomes a homogenous brown solution. While stirring at 50° C., crystallization of a product slowly begins.

After stirring for about 1 hour, the heat is removed and the reaction mixture is allowed to cool to room temperature with stirring (overnight). A brown suspension is obtained. The mixture is filtered and washed with 2-butanone under vacuum to yield 1.7 grams of an off-white to brownish powder (about 62% yield). The powder slowly turns purple at room temperature. The XRPD pattern is shown in FIG. 1. XRPD analysis shows sharp peaks, characteristic of a good crystalline material, but with some amorphous background present. ¹H-NMR is consistent with a monotosylate salt (1:1 molar ratio of tolyl protons to free base protons). The XRPD Peak List for FIG. 1 is shown below:

| | Angle | d Value | Rel. Intensity |
|---|---|---|---|
| Index | (° 2Theta) | (Å) | (%) |
| 1 | 5.877 | 15.02674 | 7.20 |
| 2 | 8.627 | 10.24128 | 1.50 |
| 3 | 11.437 | 7.73074 | 4.20 |
| 4 | 13.545 | 6.53209 | 10.80 |
| 5 | 14.933 | 5.92793 | 89.70 |
| 6 | 16.975 | 5.21919 | 63.00 |
| 7 | 19.371 | 4.5787 | 100.00 |
| 8 | 19.957 | 4.44545 | 30.60 |
| 9 | 20.94 | 4.23896 | 24.20 |
| 10 | 22.592 | 3.93251 | 18.80 |
| 11 | 23.807 | 3.7346 | 9.90 |
| 12 | 24.567 | 3.62067 | 44.00 |
| 13 | 25.342 | 3.51164 | 15.90 |
| 14 | 25.606 | 3.47614 | 16.90 |
| 15 | 25.944 | 3.4316 | 11.70 |
| 16 | 27.532 | 3.23719 | 11.70 |
| 17 | 28.041 | 3.17951 | 1.80 |
| 18 | 28.968 | 3.07987 | 3.60 |
| 19 | 29.943 | 2.98173 | 6.00 |
| 20 | 32.527 | 2.75052 | 4.60 |
| 21 | 33.612 | 2.66415 | 2.80 |
| 22 | 36.548 | 2.45664 | 3.60 |
| 23 | 36.588 | 2.45405 | 4.30 |
| 24 | 36.615 | 2.45229 | 3.80 |

The TGA/DSC thermogram of the product is shown in FIG. 3. TGA/DSC shows a gradual mass loss of about 3.7 wt. % in the temperature range of 29° C. to 198° C. A desolvation event is observed at a peak temperature of about 84° C., and a melting event is observed at peak temperature of about 180° C.

### Example 4: Co-Crystal Screen

A co-crystallization is conducted using amino acids as a co-former. 18 amino acids and four solvents are studied (methanol, DMSO, acetone, acetonitrile). A saturated solution of Compound A free base in water and a saturated solution of the co-former in water are prepared and mixed together. The maximum concentration is set to 40 mg/mL. The saturated solutions are mixed in a 1:1 ratio (by volume) and the combined solutions are shaken at 50° C. for about 16 hours. Any obtained solids were analyzed by XRPD. If XRPD showed a new pattern the solid was also characterized by FT-IR.

Initial results show the formation of new crystalline solids (by XRPD) in 24 of the 72 experiments. Crystalline solids are obtained only in experiments using DMSO, acetone, or acetonitrile as the solvent. FTIR analysis of the obtained solids shows no co-crystal formation in eight of the experiments, co-crystal formation in twelve of the experiments, and unclear results in the remaining four experiments. The results suggest co-crystals formed using cysteine, methionine, phenylalanine, serine, alanine, histidine, glycine, and valine. Questionable results were obtained for lysine and threonine as well.

The positive and questionable co-crystal experiments are repeated at a 100 mg scale. Saturated solutions of Compound A free base in DMSO, acetone, and acetonitrile are prepared (maximum concentration 40 mg/mL), and saturated solutions of the co-formers are also made with water as the media (maximum concentration 40 mg/mL). 1.5 mL of the saturated Compound A solution and 1.5 mL of the saturated co-former solution are mixed together and shaken at 50° C. for 24 hours.

It is found that only the experiments using DMSO result in formation of a solid, the other experiments resulting in a clear solution. However, further analysis of the solids by FTIR and DSC/TGA suggest that they are DMSO solvates not co-crystals. The clear solutions were then evaporated to dryness to provide solids which were analyzed by XRPD and FTIR. The results are consistent with possible co-crystal formation using alanine (acetone solvent), phenylalanine (acetone or acetonitrile solvent), and histidine (acetone or acetonitrile solvent). Further characterization of the products by DSC/TGA and 1H-NMR confirms formation of co-crystals for alanine and phenylalanine, but not histidine. Data provided in the following table:

| | | | TGA mass | DSC Tpeak | H-NMR |
|---|---|---|---|---|---|
| Solvent | Co-Former | Appearance | loss (%) | (° C.) | ratio (FB:CI) |
| Acetone | Alanine | Brownish | 1.1 | 148.0 | ~4:1 |
| | | solid | 39.7 | 278.0 | |
| Acetone | Phenylalanine | Off-white | 1.6 | 145.0 | ~1:1 |
| | | solid | 26.3 | decomp. | |
| Acetonitrile | Phenylalanine | Off-white | 3.1 | 148.0 | ~1:2 |
| | | solid | 28.8 | 251.8 | |

Additional experiments are conducted using alanine and phenylalanine with 50 mg of Compound A free base and a 1:1 molar ratio. Acetone-water (1:1, 2:1, and 3:1) and acetonitrile-water (1:1, 2:1, and 3:1) are tested in an effort to promote crystallization of the products. The reactions are stirred in 1.2 mL of the solvent mixture at 50° C. for overnight. Most of them appeared as a clear solution again, but upon cooling to about 7° C., precipitation occurs. The solids were analyzed by XRPD. New co-crystal patterns are obtained using both amino acids in most of the solvent mixtures. The data suggests that multiple polymorphic forms are available, however the thermodynamically most stable form is not determined.

### Example 5: Toluenesulfonate Salt Process Optimization

Further optimization of the process for preparation of the Compound A toluenesulfonic acid salt crystal is performed. The effect of solvent, stoichiometry, and temperature is evaluated. Considerations taken into account include the appearance and form of the product (including XRPD), yield, product purity (HPLC, ¹H-NMR), and residual solvent levels (GC-HS).

The procedure according to Example 3 is repeated for consistency (Experiment 3-2 in the table below). For the other experiments, the following general procedure is used: Compound A in free base form is added to a reactor, followed by acetone solvent. The reactor is brought to the desired initial temperature, and then a solution of p-toluenesulfonic acid monohydrate in acetone is added slowly. The reactor is continued to stir while the temperature is reduced to the desired final temperature. Crystalline product forms during this time. The mixture resulting suspension is filtered, and the filter cake is washed with acetone solvent, followed by drying.

It is found that adding the toluenesulfonic acid monohydrate as a solution, rather than as a solid, using a lower temperature, and using acetone rather than 2-butanone, results in improved results. Some of the results are summarized in the following table:

| | 3-2 | 5-1 | | 5-2 | | 5-3 | | 5-4 | | 5-5 | |
|---|---|---|---|---|---|---|---|---|---|---|---|
| Compound A- | No | No | | No | | Yes | | Yes | | Yes | |
| Recrystallized? | | | | | | | | | | | |
| Solvent | 2-butanone | Acetone | | Acetone | | Acetone | | 1:1 MTBE/ | | 1:1 MTBE/ | |
| | | | | | | | | Acetone | | Acetone | |
| p-TSA | 1.5 eq. | 0.94 eq. | | 0.94 eq. | | 0.98 eq. | | 0.98 eq | | 0.98 eq. | |
| p-TSA Form | Solid | Solution | | Solution | | Solution | | Solution | | Solution | |
| Initial Temp | 50° C. | 15° | C. | 15° | C. | 15° | C. | 15° | C. | 15° | C. |
| Final Temp | 20° C. | 0° | C. | 0° | C. | 0° | C. | 0° | C. | 0° | C. |

| | | | | | | |
|---|---|---|---|---|---|---|
| Yield | 60% | 75% | 82% | 89% | 92% | 96% |
| Colorization | Yes | No | No | No | No | Slight |
| HPLC purity | 99% | 98% | n.a. | 99% | 99% | n.a. |
| Residual acetone, ppm | n.a. | 533 | n.a. | 2993 | <250 | 703 |
| Residual MTBE, ppm | n.a. | n.a. | n.a. | n.a. | 4037 | 2979 |

The salt products obtained using the revised conditions (5-1 to 5-5) are off white to pale yellow, and they are stable in air and do not undergo color changes. The salts are tested under accelerated aging conditions of 70° C. for 3 hours to confirm chemical stability.

In further experiments, the reaction volume is increased. It is found that improved results are obtained when the initially formed filter cake is washed with cold acetone solvent, and with a final temperature of 5° C. HPLC analysis for the presence of the synthetic reagent 1-(3-chloropropoxy)-4-fluorobenzene is included in the testing. Additional results are summarized in the following table.

| | 5-6 | 5-7 | 5-8 | 5-9 |
|---|---|---|---|---|
| Solvent | Acetone | Acetone | Acetone | Acetone |
| p-TSA | 0.98 eq. | 0.98 eq. | 0.98 eq. | 0.98 eq. |
| p-TSA Form | Solution | Solution | Solution | Solution |
| Initial Temp | 15° C. | 15° C. | 15° C. | 15° C. |
| Final Temp | 15° C. | 15° C. | −5° C. | 5° C. |
| Yield | 87% | 88% | 91% | 90% |
| HPLC purity | 99% | 99% | 99% | 99% |
| Residual acetone, ppm | 568 | 158 | 217 | 30 |
| Water content | 0.6% | 0.6% | 0.8% | 1.0% |
| 1-(3-Cl-propoxy)-4-F-benzene | <10 ppm | <10 ppm | <10 ppm | <10 ppm |

The optimized procedure is then performed on a 90 g scale in a 1-L reactor equipped with a mechanical stirrer and a thermometer under nitrogen atmosphere. 90 g of Compound A free base (recrystallized from acetone-methanol) is suspended in 330 mL of acetone at 10° C. A solution of p-toluenesulfonic acid in acetone (0.98 eq; 32.98 g in 120 mL acetone) is added dropwise, while the reactor temperature is maintained at 10 to 15° C. Total acetone volume is 450 mL (0.2 g/mL for Compound A free base). The reaction is stirred for 16 hours at about 10° C. The precipitated crystalline product is filtered, and the filter cake is washed with acetone (3×110 mL). The product is dried under vacuum at 50° C. to provide 118.2 g of product (90.5% yield).

The product from this procedure is tested using ESI/LCMS, 1D and 2D NMR, elemental analysis, HPLC, FTIR, XRPD, single crystal XRD, DSC, TGA, and complete impurity analysis, including process-related impurities, heavy metals, and solvent. All test results are consistent with the desired product in high purity. Single crystal x-ray diffraction demonstrates that the crystals have monoclinic form. FIG. 4 shows the XRPD spectrum for the product from the 90-g scale reaction. The peak list for the XRPD spectrum of FIG. 4 is shown in the following table:

XRPD (Cu anode, Ni filter) for p-Tosylate Salt
Crystal (process scale up)

| # | Angle | d Value | Rel. Intensity |
|---|---|---|---|
| 1 | 2.8879 | 30.59400 | 59.95 |
| 2 | 5.6562 | 15.62516 | 100.00 |
| 3 | 8.4644 | 10.44645 | 8.16 |
| 4 | 11.2928 | 7.83563 | 26.92 |
| 5 | 13.4082 | 6.60377 | 9.39 |
| 6 | 14.8248 | 5.97578 | 78.72 |
| 7 | 16.6241 | 5.33282 | 16.49 |
| 8 | 16.9582 | 5.22852 | 90.26 |
| 9 | 18.4273 | 4.81487 | 5.19 |
| 10 | 18.6759 | 4.75131 | 19.75 |
| 11 | 19.1869 | 4.62591 | 32.80 |
| 12 | 19.4590 | 4.56184 | 18.15 |
| 13 | 19.8260 | 4.47822 | 48.35 |
| 14 | 20.2312 | 4.38943 | 6.11 |
| 15 | 20.8301 | 4.26457 | 16.06 |
| 16 | 22.1645 | 4.01075 | 3.91 |
| 17 | 22.5080 | 3.95030 | 10.90 |
| 18 | 22.6768 | 3.92128 | 9.65 |
| 19 | 23.3069 | 3.81667 | 6.41 |
| 20 | 23.6713 | 3.75874 | 9.16 |
| 21 | 24.5094 | 3.63207 | 25.32 |
| 22 | 25.0812 | 3.55056 | 6.40 |
| 23 | 25.5465 | 3.48693 | 24.59 |
| 24 | 25.8673 | 3.44441 | 6.35 |
| 25 | 26.8693 | 3.31820 | 4.06 |
| 26 | 27.4048 | 3.25187 | 11.53 |
| 27 | 27.4701 | 3.24697 | 14.54 |
| 28 | 27.9988 | 3.18686 | 3.56 |
| 29 | 28.4245 | 3.14009 | 2.31 |
| 30 | 28.8342 | 3.09640 | 2.51 |
| 31 | 29.2912 | 3.04911 | 2.79 |
| 32 | 29.9137 | 2.98706 | 2.90 |
| 33 | 31.3520 | 2.85324 | 5.46 |
| 34 | 32.4971 | 2.75526 | 3.68 |
| 35 | 33.7432 | 2.65632 | 3.64 |
| 36 | 34.2165 | 2.62065 | 2.52 |
| 37 | 36.5083 | 2.46123 | 6.15 |
| 38 | 37.3422 | 2.40816 | 1.74 |
| 39 | 37.9031 | 2.37381 | 2.00 |
| 40 | 39.2907 | 2.29311 | 1.56 |

FIGS. 5 and 6 show, respectively, the DSC and TGA thermograms from the 90-g scale reaction. The DSC thermogram shows a first endothermic phenomenon starting from 163° C., which is overlapped by a fast exothermic phenomenon (solid-solid change based on camera images). The shape of this endo-exo curve is consistent with a melting then sudden recrystallization. In the TGA test, there is no mass loss in this temperature range, consistent with recrystallization. A second endothermic phenomenon (melting) is visible between 209° C. and 230° C. (peak at 226° C.; heat: −61 J/g). At a higher temperature, from 254° C., a further exothermic phenomenon (decomposition) is detected. As the phase transition of the product is non-congruent, the melting point is to be considered as the beginning of melting (163° C.). TGA shows one mass loss step, beginning at 250° C.

This 90-g scale procedure is then performed on a 190 g scale in a 2-L reactor equipped with a mechanical stirrer and a thermometer under nitrogen atmosphere. At the larger scale, a relatively larger volume of acetone is used to suspend the Compound A free base, and a smaller volume of acetone is used for the p-toluenesulfonic acid. The cooling crystallization and drying conditions are also improved.

190 g of Compound A free base (recrystallized from acetone-methanol) is suspended in 1700 mL of acetone at 10° C. A solution of p-toluenesulfonic acid in acetone (0.98 eq; 92.85 g in 200 mL acetone) is added dropwise, while the reactor temperature is maintained at 10 to 15° C. Total acetone volume is 1900 mL (0.1 g/mL for Compound A free base). The reaction is stirred for 3 hours at about 10° C., and then cooled to 5° C. over 1.5 hours, and then stirred at 5° C. for 8 hours. The precipitated crystalline product is filtered, and the filter cake is washed with acetone (3×200 mL). The product is dried under vacuum at 40° C. to provide 249.5 g of product (90.5% yield). Characterization of the two products provides the following information:

| | | |
|---|---|---|
| Parameter | 90 g scale | 190 g scale |
| Appearance | Light brown powder | Pale yellow powder |
| HPLC purity | 99% | 99% |
| Water content | 0.8% | 0.6% |
| Residual solvents: | | |
| Methanol | <150 ppm | <150 ppm |
| Acetone | 375 ppm | 1046 ppm |
| Acetonitrile | <21 ppm | <21 ppm |
| MTBE, EtOAc, | Each <250 ppm | Each <250 ppm |
| Heptane, DMSO | | |
| Toluene, DMF, | Each <60 ppm | Each <60 ppm |
| DMA | | |
| 1-(3-Cl-propoxy)-4- | | <10 ppm |
| F-benzene | | |

The 190-g scale procedure is repeated successfully on a scale of 5.2 kg Compound A free base in a 160-L glass-lined reactor. At this larger scale, additional acetone is used to wash the reactor feeding lines, but the final concentration is the same, 0.1 g/mL. Addition of the p-toluenesulfonic acid is carried out over about 40 minutes at 10-15° C., followed by stirring for 3 hours at same, cooling to 5° C. (over about 1.5-2 hours), and then stirring at 5° C. for 14 hours. Drying is carried out under vacuum at a temperature up to 40° C. Net yield is 78%, mainly due to product loss stuck to the reactor walls and filter dryer. The following analytical results are obtained for the product:

| | |
|---|---|
| Parameter | 5.2 kg scale |
| Appearance | Pale yellow powder |
| HPLC purity | 99% |
| Water content | 0.6% |
| Residual solvents: | |
| Methanol | <150 ppm |
| Acetone | 2869 ppm |
| Acetonitrile | <21 ppm |
| MTBE, EtOAc, | Each <250 ppm |
| Heptane, DMSO | |
| Toluene, DMA | Each <60 ppm |
| 1-(3-Cl-propoxy)-4-F-benzene | <10 ppm |
| Methyl p-Toluenesulfonate | <20 ppm |
| Ethyl p-Toluenesulfonate | <10 ppm |
| Isopropyl p-Toluenesulfonate | <10 ppm |
| Related substances impurities | Net 0.4% |
| Synthetic intermediates | <0.05% |
| Synthetic side-products | <0.20% |
| Heavy Metals: | |
| Copper | <10 ppm |
| Nickel, Lithium, Antimony | Each <10 ppm |
| As, Cd, Co, Pb, Hg, V, | Each <1 ppm |

The examples set forth hereinabove are intended illustrate the invention but should not be interpreted as a limitation thereon.

All of the references cited hereinbefore are hereby incorporated in reference in their entirety.

1. (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one in solid, crystalline salt form, wherein the salt form is a p-toluenesulfonate salt, and wherein the salt is chemically stable in air, or
(6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one in solid, crystalline salt form, wherein the salt form is selected from a hydrochloride, a tartrate, a malate, a fumarate, a glutamate, an oxalate, a besylate, and an ascorbate.

2. The p-toluenesulfonate salt according to claim 1, wherein the salt has a crystal structure characterized by an X-ray powder diffraction pattern having at least 5, or at least 6, or at least 7, or at least 8, peaks having angle (2-theta) values selected from the group consisting of about 5.9, 8.6, 11.4, 13.6, 14.9, 17.0, 19.4, 20.0, 20.9, 22.6, 23.8, 24.6, 25.3, 25.6, 25.9, 27.5, 28.0, 29.0, 29.9, 32.5, 33.6, 36.6, 36.6, and 36.6, taking into account potential variations due to sample purity and instrument variation, e.g., wherein the X-ray powder diffraction pattern is generated using an X-ray diffractometer with a copper anode and a nickel filter.

3. The (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one solid, crystalline salt according to claim 1, wherein the salt form is selected from a hydrochloride, a tartrate, a malate, a fumarate, a glutamate, an oxalate, a besylate, and an ascorbate.

4. The salt according to claim 1, wherein the salt is at least 98% pure.

5. The salt according to claim 1, wherein the salt comprises less than 1 wt. % of (6bR,10aS)-8-(3-(4-fluorophenoxy)propyl)-6b,7,8,9,10,10a-hexahydro-1H-pyrido[3′, 4′: 4,5]pyrrolo[1,2,3-de]quinoxalin-2(3H)-one in free base form.

6. The salt according to claim 1, wherein in the salt comprises less than 1 wt. % of any p-toluenesulfonic acid, tartaric acid, malic acid, fumaric acid, glutamic acid, oxalic acid or ascorbic acid.

7. The salt according to claim 1, wherein the salt comprises less than 0.5 wt. %, or less than 0.1 wt. %, of any synthetic precursor of, or side-product from, the synthetic method used to prepare the (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one in free base form.

8. The salt according to claim 1, wherein the salt comprises less than 5000 ppm of all residual solvents.

9. The salt according to claim 1, wherein the salt comprises less than 100 ppm of 1-(3-chloropropoxy)-4-fluorobenzene.

10. The salt according to claim 1, wherein the salt comprises less than 1 wt. % of water.

11. The salt according to claim 1, wherein the salt comprises less than 100 ppm of p-toluenesulfonate alkyl esters (e.g., methyl, ethyl and/or isopropyl esters).

12. A method of making (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one in solid, crystalline p-toluenesulfonic acid salt form, comprising:
(a) reacting (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one free base with p-toluenesulfonic acid together with an organic solvent comprising acetone; and
(b) recovering the salt thus formed.

13. A method of making the salt according to claim 3, comprising:
(a) reacting (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one free base with an acid selected from hydrochloric acid, tartaric acid, malic acid, fumaric acid, glutamic acid, oxalic acid, benzenesulfonic acid, and ascorbic acid, e.g., together with an organic solvent; and
(b) recovering the salt thus formed.

14. (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one in solid, co-crystal form.

15. The co-crystal of claim 14, wherein the co-crystal is a co-crystal with an amino acid (e.g., a natural alpha-amino acid), for example, an amino acid is selected from alanine, cysteine, methionine, phenylalanine, serine, glycine, histidine, and valine.

16. A method of making the co-crystal according to claim 14, comprising:
(a) reacting (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one free base with a crystal co-former, optionally selected from an amino acid selected from alanine, asparagine, cysteine, methionine, phenylalanine, proline, serine, glutamine, glycine, histidine, lysine, threonine, tyrosine, and valine (e.g., alanine or phenylalanine), for example, together with an organic solvent (e.g., selected from methanol, dimethyl sulfoxide, acetone, or acetonitrile, e.g., acetone or acetonitrile); and
(b) recovering the co-crystal thus formed.

17. A pharmaceutical composition comprising the salt according to claim 1, or a solid co-crystal of (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one, in combination or association with a pharmaceutically acceptable diluent or carrier.

18. A method for the prophylaxis or treatment of a human suffering from a disease or abnormal condition involving or mediated by the 5-HT_{2A} receptor, serotonin transporter (SERT), dopamine D₁/D₂ receptor signaling pathways, and/or the mu-opioid receptor, the method comprising administering to a patient in need thereof a therapeutically effective amount of a salt according to claim 1 or a solid co-crystal of (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one.

19. The method according to claim 18, wherein the disease or abnormal condition is a disorder selected from obesity, anxiety, depression (for example refractory depression and MDD), psychosis (including psychosis associated with dementia, such as hallucinations in advanced Parkinson's disease or paranoid delusions), schizophrenia, sleep disorders (particularly sleep disorders associated with schizophrenia and other psychiatric and neurological diseases), sexual disorders, migraine, conditions associated with cephalic pain, social phobias, agitation in dementia (e.g., agitation in Alzheimer's disease), agitation in autism and related autistic disorders, gastrointestinal disorders such as dysfunction of the gastrointestinal tract motility, and dementia, for example dementia of Alzheimer's disease or of Parkinson's disease; mood disorders; and drug dependencies, for example, opiate or opioid dependency and/or alcohol dependency, or withdrawal from drug or alcohol dependency (e.g., opiate or opioid dependency); or binge eating disorder; or opiate or opioid overdose; or opioid use disorder (OUD); or substance use disorder or substance abuse disorder (e.g., as these terms are defined in the DSM-V), optionally in patient also suffering from anxiety and/or depression.

20. The method according to claim 18, wherein the disease or abnormal condition is a disorder selected from obsessive-compulsive disorder (OCD), obsessive-compulsive personality disorder (OCPD), general anxiety disorder, social anxiety disorder, panic disorder, agoraphobia, compulsive gambling disorder, compulsive eating disorder, body dysmorphic disorder, hypochondriasis, pathological grooming disorder, kleptomania, pyromania, attention deficit-hyperactivity disorder (ADHD), attention deficit disorder (ADD), impulse control disorder, and related disorders, and combinations thereof; or a pain disorder, e.g., a condition associated with pain, such as cephalic pain, idiopathic pain, neuropathic pain, chronic pain (e.g., moderate to moderately severe chronic pain, for example, in patients requiring 24-hour extended treatment for other ailments), fibromyalgia, dental pain, traumatic pain, or chronic fatigue; or wherein the central nervous system disease or disorder is a drug dependency (for example, opiate or opioid dependency (i.e., opioid use disorder), cocaine dependency, amphetamine dependency, and/or alcohol dependency), or withdrawal from drug or alcohol dependency (e.g., opiate, opioid, cocaine, or amphetamine dependency), and wherein the patient also suffers from a co-morbidity, such as anxiety, depression or psychosis, and/or wherein the patient also suffers from an opiate or opioid overdose.

21. The method according to claim 19c, wherein the patient suffers from a pre-existing or co-morbid gastrointestinal disorder and/or pulmonary disorder, for example, wherein the pre-existing or co-morbid disorder is selected from the group consisting of irritable bowel syndrome, pelvic floor disorder, diverticulitis, inflammatory bowel disease, colon or colorectal cancer, celiac disease, non-celiac gluten sensitivity, asthma, chronic obstructive pulmonary disease (COPD), dyspnea, pneumonia, congestive heart failure, interstitial lung disease, pneumothorax, bronchitis, pulmonary embolism, and traumatic chest injury (e.g., broken sternum or ribs, bruised intercostal muscles).
